(19) United States
(12) Patent Application Publication
(10) Pub. No.: US 2024/0390385 A1
(43) Pub. Date: Nov. 28, 2024

(54) Composition and Methods for the Prevention of Aminoglycoside Toxicity

(71) Applicant: Ting Therapeutics LLC, La Jolla, CA (US)

(72) Inventors: Jian Zuo, Carlsbad, CA (US); Marisa Laura Zallocchi, Omaha, NE (US)

(73) Assignee: Ting Therapeutics LLC, La Jolla, CA (US)

(21) Appl. No.: 18/792,993

(22) Filed: Aug. 2, 2024

Related U.S. Application Data
(63) Continuation of application No. 18/778,972, filed on Jul. 20, 2024.
Application No. 18/778,972 is a continuation of application No. 18/031,998, filed on Apr. 14, 2023, now Pat. No. 12,070,465.
(60) Provisional application No. 63/078,571, filed on Sep. 15, 2020.

(51) Int. Cl. A61K 31/55; A61K 31/397; A61K 31/45; A61P 27/16

(52) U.S. Cl. CPC A61K 31/55; A61K 31/397; A61K 31/45; A61P 27/16

(57) ABSTRACT

The present invention relates to therapeutic uses of active agents such as for treating, inhibiting, and/or preventing damage associated with aminoglycoside toxicity. The data establishes that piperlongumine can protect hair cells from aminoglycoside damage within a wide range of concentrations. The range is 0.1 pM to 25 uM. Furthermore, piperlongumine protects renal function, it has a good therapeutic index (TI>200,000), no interference with AG's anti-bacterial activity, and no drug-drug interaction. This abstract is intended as a scanning tool for purposes of searching in the particular art and is not intended to be limiting of the present invention.

### CROSS-REFERENCE TO RELATED APPLICATIONS

This application claims the benefit of U.S. patent application Ser. No. 18/031,998 filed Apr. 14, 2023, pct/us21/50425 filed Sep. 16, 2021, U.S. provisional patent application Ser. No. 63/078,571 filed Sep. 15, 2020, under 35 USC § 119(e) and 35 U.S.C. § 111(a) (hereby specifically incorporated herein by reference).

### STATEMENT REGARDING FEDERALLY SPONSORED RESEARCH OR DEVELOPMENT

This invention was made with government support under R01DC015010 R01DC015444, R43DC019065 from the NIH, N00014-18-1-2507 from the Navy/ONR, and W81XWH-18-1-0442 from the Army/MRMC. The government has certain rights in the invention.

### BACKGROUND OF THE INVENTION

### (1) Field of the Invention

The present invention relates to therapeutic uses of active agents such as for treating, inhibiting, and/or preventing damage associated with aminoglycoside toxicity.

### (2) Description of Related Art Including Information Disclosed Under 37 CFR 1.97 and 37 CFR 1.98

Over 35 million Americans suffer from hearing impairment. In mammals, hair cell impairment is permanent. Antibiotic-induced hearing impairment (AIHL) is a permanent side effect from often necessary medical care. Neonates admitted to the neonatal intensive care unit have ten times the rate of hearing loss as neonates not admitted, often associated with the administration of antibiotics. The spiral-shaped cochlea of the inner ear is responsible for detecting sound. Inner hair cells lining the cochlea transform the mechanical vibrations of sound waves into chemical signals. These chemicals are then released from the hair cells and received by receptors on the auditory nerve fibers that send electrical impulses to the brain. The inner ear cells lining the cochlea can be destroyed by the use of antibiotics, leading to a condition called sensorineural hearing impairment.

Currently, there are no clinically proven medications for the treatment of hearing impairment (sensorineural and neural), or tinnitus associated with the inner ear, and a medication that could be used to prevent, alleviate, or eliminate hearing impairment (or tinnitus) would thus be very desirable. The most common remedy for individuals suffering from severe sensorineural hearing impairment is a hearing aid, which functions to amplify sound. Hearing aids are non-invasive and can improve an individual's ability to hear. However, hearing aids can often be quite conspicuous and embarrassing to the wearer and hearing aids do not return hearing to normal levels. Furthermore, hearing aids amplify sound indiscriminately, sometimes amplifying sounds that an individual does not wish to hear, such as environmental noise. Du has shown that ethanol extracts of a piper longum L. attenuates gentamicin-induced hair cell loess in neonatal cochlea cultures. See e.g KR10-2013-0004949 and KR-2010-016925-2. However, neither ethanol nor hexane can be systemically administered to a patient. There exists a need in the art for a practical solution to prevent aminoglycoside toxicity affecting both inner ear cells and renal tissue.

### BRIEF SUMMARY OF THE INVENTION

The inventive subject matter includes a method to prevent aminoglycoside toxicity by systemically co-administering to an animal or human, an aminoglycoside and an effective amount of piperlongumine to prevent aminoglycoside toxicity. Here the aminoglycoside toxicity can include aminoglycoside toxicity affects ear hair cells and renal function. Piperlongumine can prevent aminoglycoside toxicity in ear hair cells and renal tissue. Aminoglycosides include but are not limited to gentamicin, tobramycin, amikacin, neomycin, netilmicin, plazomicin, paromomycin, streptomycin and kanamycin

The inventive subject matter includes a method of preventing aminoglycoside toxicity, the method including the steps of administering a therapeutically effective amount of an active agent to downregulate TRPV1, or a pharmaceutically acceptable salt thereof and an aminoglycoside, wherein the active agent is piperlongumine, a derivative of piperlongumine or a pharmaceutically acceptable salt thereofThe inventive subject matter further includes: a formulation for administration to a subject made of an effective amount of a derivative of piperlongumine to prevent aminoglycoside toxicity in a pharmaceutically acceptable carrier, wherein the aminoglycoside toxicity affects ear hair cells or wherein the aminoglycoside toxicity affects renal function. The term derivative means: a compound that is derived from a similar compound by a chemical reaction or a structural analogue of piperlongumine. The inventive subject matter further includes a kit made of an active agent, wherein the active agent is a derivative of piperlongumine; or a pharmaceutically acceptable salt thereof; and one or more of: at least one antibiotic; at least one cancer drug; and instructions for preventing a hearing impairment. Similarly, the inventive subject matter includes: a kit including an active agent, wherein the active agent is piperlongumine, a derivative of piperlongumine, or a pharmaceutically acceptable salt thereof; and one or more of: at least one aminoglycoside antibiotic; and instructions for preventing aminoglycoside toxicity.

### BRIEF DESCRIPTION OF THE SEVERAL VIEWS OF THE DRAWING

The accompanying figures, which are incorporated in and constitute a part of this specification, illustrate several aspects and together with the description serve to explain the principles of the invention. The patent or application file contains at least one drawing executed in color. Copies of this patent or patent application publication with color drawing(s) will be provided by the Office upon request and payment of the necessary fee.

FIG. 1A is an illustration showing (E)-1-(3-(3,4,5-trimethoxyphenyl)acryloyl)-S,6-dihydropyridin-2(1H)-one (piperlongumine) having a structure represented by the formula as shown.

FIG. 1B is an illustration showing the formula of derivatives of piperlongumine.

FIG. 2 illustrates a synthetic scheme to make the derivatives of this invention.

FIG. 3 illustrates a synthetic scheme to make the derivatives of this invention.

FIG. 4A illustrates synthetic schemes to make the derivatives of this invention.

FIG. 4B illustrates synthetic schemes to make the derivatives of this invention.

FIG. 5A shows that piperlongumine protects zebrafish neuromasts from neomycin-induced hair cell death.

FIG. 5B shows that piperlongumine protects zebrafish neuromasts from neomycin-induced hair cell death.

FIG. 5C shows that piperlongumine protects zebrafish neuromasts from gentamycin-induced hair cell death.

FIG. 6 shows piperlongumine derivatives protect zebrafish neuromasts from excitotoxic damage. 5 dpf Tg (Brn3c:mGFP) larvae were used for the experiments. Zebrafish were pretreated with kainic acid for one hour (300 μM) to mimic the excitotoxic damage induced by noise exposure, and then, post-treated with piperlongumine-25 for 2 hours (at concentrations of 0.001 μM; 0.1 μM; 10 μM; and 100 μM). Control animals were treated with DMSO (−) or piperlongumine-25 alone (+). Animals were then fixed and immunostained for GFP. Quantification was done by assessing three neuromasts in the same anatomic location of each animal (n=5). The data is plotted as mean+SD. Statistical testing was done with One-way ANOVA and Dunnett post-hoc test (P<0.0001 compared to KA).

FIG. 7 shows protection of mouse embryonic fibroblast (MEFs) by piperlongumine against aminoglycosides. MEFs were incubated for 15 hours with neomycin (100 μM) or gentamicin (8 μM) with or without piperlongumine (0.1 nM-5 μM). Cell viability was assessed employing MTT (3-(4-,5-dimethylthiazol-2-yl)-2,5-diphenyltetrazolium bromide) viability kit test (Thermo Fisher). The data is plotted as mean+SD. Statistical testing was done with One-way ANOVA and Dunnett post-hoc test (*P<0.05, **P<0.01 compared to either Neo or GM, respectively).

FIG. 8A shows A (E)-1-(3-(3,4,5-trimethoxyphenyl)acryloyl)-S,6-dihydropyridin-2(1H)-one preserves normal hair cell function in antibiotic exposed mice. C57BI6 (Cdh23 corrected) (6-7 weeks old) were exposed to vehicle+kanamycin (Veh+Kan) (700 mg/kg b.w. twice a day for 14 days) (shown as circles with four breaks); or (E)-1-(3-(3,4,5-trimethoxyphenyl)acryloyl)-S,6-dihydropyridin-2(1H)-one+kanamycin (PLM+Kan) with (E)-1-(3-(3,4,5-trimethoxyphenyl)acryloyl)-S,6-dihydropyridin-2(1H)-one (40 mg/kg b.w. once a day for 17 days) (shown as circle no breaks). Age match controls are shown (age, shown as circle with three breaks). Auditory brainstem response (ABR) thresholds were recorded for frequencies (4 kHz, 5.6 kHz, 8 kHz, 16 kHz, 22.6 kHz, 32 kHz, 45.2 kHz, and 64 kHz) before and after treatment protocol. We observed significant protection when animals were co-treated with PLM at 22.6 KHZ compared to animals' kanamycin alone. Statistical analysis was done with repeated measures ANOVA between groups.

FIG. 8B shows piperlongumine (E)-1-(3-(3,4,5-trimethoxyphenyl)acryloyl)-S,6-dihydropyridin-2(1H)-one is not toxic with and without kanamycin exposure in C57BI6 mice in vivo over the 17-day treatment regimen.

FIG. 9A shows piperlongumine (E)-1-(3-(3,4,5-trimethoxyphenyl)acryloyl)-S,6-dihydropyridin-2(1H)-one is not toxic with and without kanamycin exposure in C57BI6 mice in vivo over the 17-day treatment regimen.

FIG. 9B shows piperlongumine (E)-1-(3-(3,4,5-trimethoxyphenyl)acryloyl)-S,6-dihydropyridin-2(1H)-one and its derivatives reduce NF-kB activity in zebrafish.

FIG. 10 shows piperlongumine derivatives were tested in a zebrafish model for excitotoxicity.

FIG. 11 shows piperlongumine derivatives PG3, PG18, PG25, PG53 and PG54 performed better than piperlongumine in a zebrafish model for excitotoxicity.

FIG. 12A Five to six days post-fertilization (dpf) zebrafish were exposed to kanamycin in the absence/presence of piperlongumine (PL). Zebrafish co-treated with piperlongumine (8 μM-8 pM) and kanamycin (400 μM) and immunostained for otoferlin. At least three neuromasts/fish were inspected (5-6 fish). Results are expressed as percentage of protection, with 100% representing vehicle-treated and 0% kanamycin-treated fish. piperlongumine-only: #1=25 μM, #2=1 μM and #3=0.1 μM. piperlongumine's chemical structure is shown with the colchicine motif in red.

FIG. 12B Five to six days post-fertilization (dpf) zebrafish were exposed to kanamycin in the absence/presence of piperlongumine. Representative images of zebrafish neuromasts. GFP positive hair cells immunostained for otoferlin (red).

FIG. 12C Five to six days post-fertilization (dpf) zebrafish were exposed to kanamycin in the absence/presence of piperlongumine. Representative images of zebrafish neuromasts. GFP-positive HCs immunostained for otoferlin (red).

FIG. 12D Five to six days post-fertilization (dpf) zebrafish were exposed to kanamycin in the absence/presence of piperlongumine. Representative images of zebrafish neuromasts. GFP-positive hair cells immunostained for otoferlin (red).

FIG. 12E Five to six days post-fertilization (dpf) zebrafish were exposed to kanamycin in the absence/presence of piperlongumine. Representative images of zebrafish neuromasts. GFP-positive HCs immunostained for otoferlin (red).

FIG. 12F a toxicity assay performed in the mouse HC line, HEI-OC1, showed that piperlongumine can kill 50% of the cells at 25.5 μM, ˜200,000-fold higher than its effective dose.

FIG. 12G shows piperlongumine treated cells were undistinguishable from control. Representative images from 3 independent experiments in HEI-OC1. Vehicle (G), PL 25 μM (H) or colchicine 10 μM (I). α-tubulin immunostaining (grey). PL 25 μM (H) or colchicine 10 μM (I). α-tubulin immunostaining (grey). J: Proximal tubule function in zebrafish (6-7 dpf) in the presence/absence of kanamycin and piperlongumine (PL1=8 μM, PL2=150 nM and PL3=150 pM). Inset: standard curve. Circles represent independent values.

FIG. 12H shows piperlongumine treated cells were undistinguishable from control. Representative images from three independent experiments in HEI-OC1. Vehicle.

FIG. 12I shows colchicine-treated cells showed a disruption of microtubule organization. Representative images from three independent experiments in HEI-OC1.

FIG. 12J shows kanamycin exposure resulted in renal proximal tubule dysfunction. The co-incubation with piperlongumine significantly reduced the amount of ½vdbp-mCherry released into the water. Proximal tubule function in zebrafish (6-7 dpf) in the presence/absence of kanamycin and piperlongumine (PL1=8 μM, PL2=150 nM and PL3=150 pM). Inset: standard curve. Circles represent independent values.).: Proximal tubule function in zebrafish (6-7 dpf) in the presence/absence of kanamycin and piperlongumine (PL1=8 μM, PL2=150 nM and PL3=150 pM). Inset: standard curve. Circles represent independent values.

FIG. 12K shows drug-drug interactions (DDI) experiments showed that piperlongumine (1 mM-0.32 mM) did not have any effect on CYP450's activity in vitro.DDI studies. Ketoconozale: negative control. Results are presented as the average fluorophore concentration. Scale bar: B-E=5 μm, G-I=10 μm.Mean+/−SEM. Brown-Forsythe and Welch ANOVA followed by Dunnett T3 post-test for multiple comparisons. *P<0.05, *P<0.01 versus KM400 (J) or DMSO (K).

FIG. 13A shows piperlongumine protects from AGIHL. 7-8 weeks old mice treated with vehicle (black), kanamycin (KM600, red), kanamycin+PL (KM600+PL, blue) or PL (green). ABR thresholds at 4 kHz to 64 kHz frequencies measured before (baseline, grey) and 4 weeks post-treatment.

FIG. 13B shows ABR threshold shifts. Mean+/−SEM. Circles represent individual values.

FIG. 13C shows representative ABR traces at the 22.6 kHz frequency for the four treatments. Mean+/−SEM. Circles represent individual values. Representative ABR traces at the 22.6 kHz frequency for the four treatments, DPOAEs at 4 kHz to 22.6 kHz frequencies measured before. DPOAEs at 4 kHz to 22.6 kHz frequencies measured before.

FIG. 13D shows 4 weeks post-treatment. Two-way (A, D-E) and one-way ANOVA (B) followed by Dunnett post-test for multiple comparisons. *P<0.05, **P<0.01, and ***P<0.001 control versus KM600 (A, D E,), control versus KM600+PL (A,D,E,blue), KM600 versus KM600+PL (B). The number of animals per group is between brackets.

FIG. 13E ABR threshold shifts. Mean+/−SEM. Circles represent individual values. C: Representative ABR traces at the 22.6 kHz frequency for the four treatments and 4 weeks post-treatment. Threshold averages+/−SEM.

FIG. 14 shows piperlongumine protects OHCs from AGIHL. kanamycin+PL (KM600+PL), and Ae-An: PL-only. Scale bar=10 μm. Ao: OHC quantification. Mean+/−SEM. Circles represent individual values. Two-way ANOVA followed by Dunnett post-test for multiple comparisons. ***P<0.001 versus corresponding control.

FIG. 15 shows piperlongumine protects pre-synaptic ribbons from AGIHL. kanamycin+PL (KM600+PL), and Ae-An: PL-only. Scale bar=10 μm. Ao: Pre-synaptic ribbon quantification. Mean+/−SEM. Circles represent individual values. Two-way ANOVA followed by Dunnett post-test for multiple comparisons. ***P<0.001, **P<0.01, *P<0.05 versus corresponding control.

FIG. 16 shows piperlongumine signaling after kanamycin treatment. Membranes were re-probed with anti-AKT or anti-ERK1/2. Numbers on the left indicate molecular weights. Immunoblot quantification of at least three independent experiments. B: Two-way ANOVA followed by Dunnett post-test for multiple comparisons, D: Brown-Forsythe and Welch ANOVA followed by Dunnett T3 post-test for multiple comparisons; ***P<0.001, **P<0.01 versus kanamycin.

FIG. 17 shows piperlongumine affects AKT1 phosphorylation. Quantification is presented as the ratio of fluorescence for each region relative to the total fluorescence. Mean+/−SEM. Circles represent values from different animals. Three independent samples were analyzed. One-way ANOVA followed by Dunnett post-test for multiple comparisons. *P<0.05 versus kanamycin.

FIG. 18 shows piperlongumine treatment results in TRPV1 downregulation Quantification is presented as the percentage of fluorescence for each region relative to the total fluorescence. Mean+/−SEM. Circles represent values from different animals. Three independent samples were analyzed. One-way ANOVA followed by Dunnett post-test for multiple comparisons. *P<0.05 versus kanamycin.

FIG. 19A shows piperlongumine regulates AG uptake via TRPV1 interaction. quantification of three independent immunoblots. One-way ANOVA followed by Dunnett post-test for multiple comparisons. *P<0.05 versus CP.

FIG. 19B shows quantification of three independent immunoblots. One-way ANOVA followed by Dunnett post-test for multiple comparisons. *P<0.05 versus CP.

FIGS. 19C-19R show representative images of three independent experiments in KPT2-Trpv1 (C-J,M-P) or KPT2-pBabe (K-L,TR cells. C-L: Cells incubated with media-only (C-D,K-L), capsaicin (CP, E-I), or CP+PL (G-J), and stained for pAKT1 (red), phalloidin (green) and DAPI. M-R KPT2-cells incubated with gentamicin-Texas Red (GTTR,M,R), GTTR+capsaicin (GTTR+CP,N), GTTR+CP+PL 1Mμ (O), and GTTR+capsazepine (GTTR+CPZ,P). Scale bar=20 μm.

### DETAILED DESCRIPTION OF THE INVENTION

The present invention can be understood more readily by reference to the following detailed description of the invention and the examples included therein. Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar to or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

In one aspect, compounds can be used as a therapy for the treatment and/or prevention of hearing loss. In various aspects, the compounds and compositions of the invention can be administered in pharmaceutical compositions, which are formulated according to the intended method of administration. The compounds of this invention are defined as a therapeutically active agent in a treatment regimen or procedure that is intended for preventing hearing loss by noise or aging by protecting inner ear cells from death and in preventing hearing loss by chemotherapy or antibiotics induced hearing loss. Therapeutic agent means a chemical substance that is used for the treatment or mitigation of a disease condition or ailment. In one aspect, compounds can be used as a therapy for the treatment and/or prevention of hearing loss. In various aspects, the compounds and compositions of the invention can be administered in pharmaceutical compositions, which are formulated according to the intended method of administration. The compounds of this invention are defined as a therapeutically active agent in a treatment regimen or procedure that is intended for preventing hearing loss by noise or aging by protecting inner ear cells from death and in preventing hearing loss by chemotherapy or antibiotics induced hearing loss. Therapeutic agent means a chemical substance that is used for the treatment or mitigation of a disease condition or ailment.

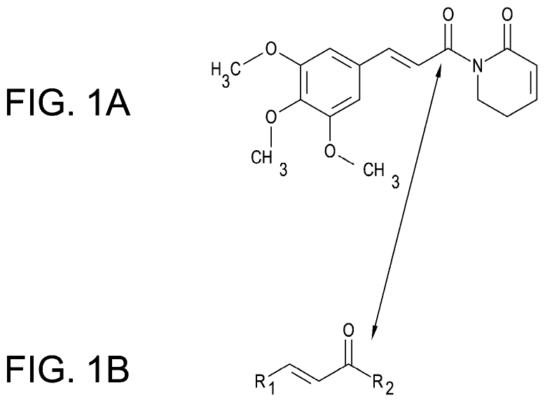

Now referring to FIG. 1, piperlongumine. (E)-1-(3-(3,4,5-trimethoxyphenyl)acryloyl)-S,6-dihydropyridin-2(1H)-one, is shown. piperlongumine is an alkaloid, which is extracted from Piper longum Linn. It exhibits anti-atherosclerotic, anxiolytic, antidiabetic, antidepressant, antibacterial, anti-platelet, aggregation, anxiolytic and anti-inflammatory properties. Piperlongumine prevents the production of tumor necrosis factor-a and interleukin-6. It is also referred to as Piplartin, 5,6-Dihydro-1-[(2E)-1-oxo-3-(3,4,5-trimethoxyphenyl)-2-propen-1-yl]-2(1H)-Pyridinone, Piperlongumine, 5,6-Dihydro-1-(1-oxo-3-[3,4,5-trimethoxyphenyl]-trans-2-propenyl)-2 [1H]-pyridinone.

A number of derivatives of piperlongumine similarly show efficacy as a therapy for the treatment and/or prevention of hearing loss. The generic structures of derivatives within the scope of this invention are shown. More specifically, acryloyl derivatives are contemplated. The acryloyl group is a form with the structure H₂C═CH—C(═O)—; it is the acyl group derived from acrylic acid.

Derivatives of piperlongumine formed from the structure:

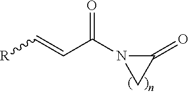

Wavy bond indicated E and Z isomers.
N=1, 2, 3, 4, 5, 6
R=H, alkyl,, hydroxy, thiol, halogen, acid, ester, amide, amine, substituted alkyl chain, substituted cyclic alkyl ring, heterocycle, piperidine, cyclohexanol, heteroaromatic, substituted heteroaromatic, cyclo alipthatic, hetero-cyclo aliphatic ring. In another aspect, derivatives of piperlongumine are formed from the structure:

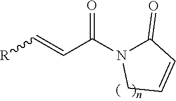

Wavy bond indicated E and Z isomers.
N=0, 1, 2, 3, 4
R=H, alkyl, alkoxy, hydroxy, thiol, halogen, acid, ester, amide, amine, substituted alkyl chain, substituted cyclic alkyl ring, heterocycle, piperidine, cyclohexano, I heteroaromatic, substituted heteroaromatic, cycloalipthatic, hetero-cyclo aliphatic ring.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6alkyl, C1-C7 alkyl, C1-C8 alkyl, C1-C9 alkyl, C1-C10 alkyl, and the like up to and including a C1-C24 alkyl. A cyclic compound (or ring compound) is a term for a compound in the field of chemistry in which one or more series of atoms in the compound is connected to form a ring. Rings may vary in size from three to many atoms, and include examples where all the atoms are carbon (i.e., are carbocycles), none of the atoms are carbon (inorganic cyclic compounds), or where both carbon and non-carbon atoms are present (heterocyclic compounds).

The base structure of derivatives of piperlongumine is shown in FIG. 1B and Table 1 is:

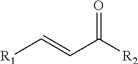

R₁=2-furanyl, 2-thiophenyl, phenyl 3-methoxyphenyl, 3-trifluromethylphenyl, 4-trifuloromethylphenyl, 4-chloro, 4-nitro, 2,2-difluoro-1,3-benzodioxole, 2-naphthyl; and

### R₂=N-linked Beta-, Gamma-, Delta-, or Epsilon-lactam.

TABLE 1

| | | | | | | NET |
|---|---|---|---|---|---|---|
| Compound | | | | | | Wt |
| ID | Structure | m/z | Mol For | IW | FW | (mg) |
| SHJ-1 | 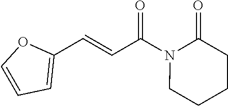 [SMILES: O=C(/C=C/c1ccco1)N1CCCCC1=O] | 219.0895 | C₁₂H₁₃NO₃ | 948.4 | 968.5 | 20.1 |
| SHJ-2 | 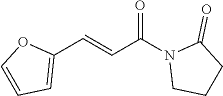 [SMILES: O=C(/C=C/c1ccco1)N1CCCC1=O] | 205.0739 | C₁₁H₁₁NO₃ | 974 | 984.1 | 10.1 |
| SHJ-3 | 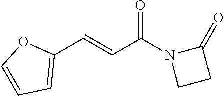 [SMILES: O=C(/C=C/c1ccco1)N1CCC1=O] | 191.0582 | C₁₀H₉NO₃ | 1005 | 1013.4 | 8.4 |
| SHJ-4 | 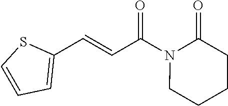 [SMILES: O=C(/C=C/c1cccs1)N1CCCCC1=O] | 235.0667 | C₁₂H₁₃NO₂S | 971.7 | 977.6 | 5.9 |
| SHJ-5 | 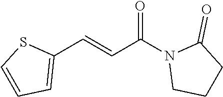 [SMILES: O=C(/C=C/c1cccs1)N1CCCC1=O] | 221.051 | C₁₁H₁₁NO₂S | 966.2 | 973.1 | 6.9 |
| SHJ-6 | 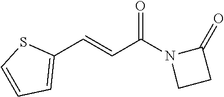 [SMILES: O=C(/C=C/c1cccs1)N1CCC1=O] | 207.0354 | C₁₀H₉NO₂S | 978 | 982.1 | 4.1 |
| SHJ-16 | 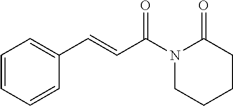 [SMILES: O=C(/C=C/c1ccccc1)N1CCCCC1=O] | 229.1103 | C₁₄H₁₅NO₂ | 942.4 | 950 | 7.6 |
| SHJ-17 | 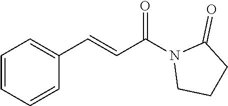 [SMILES: O=C(/C=C/c1ccccc1)N1CCCC1=O] | 215.0946 | C₁₃H₁₃NO₂ | 948.1 | 957.7 | 9.6 |
| SHJ-18 | 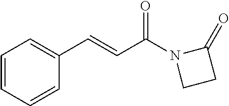 [SMILES: O=C(/C=C/c1ccccc1)N1CCC1=O] | 201.079 | C₁₂H₁₁NO₂ | 947.8 | 953 | 5.2 |
| SHJ-25 | 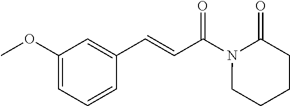 [SMILES: COc1cccc(/C=C/C(=O)N2CCCCC2=O)c1] | 259.1208 | C₁₅H₁₇NO₃ | 988.3 | 997.3 | 9 |
| SHJ-26 | 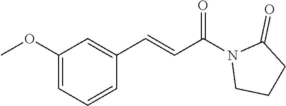 [SMILES: COc1cccc(/C=C/C(=O)N2CCCC2=O)c1] | 245.1052 | C₁₄H₁₅NO₃ | 954.5 | 962.8 | 8.3 |
| SHJ-27 | 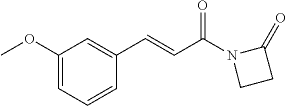 [SMILES: COc1cccc(/C=C/C(=O)N2CCC2=O)c1] | 231.0895 | C₁₃H₁₃NO₃ | 950.4 | 955.3 | 4.9 |
| SHJ-31 | 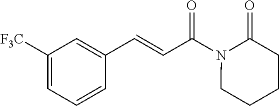 [SMILES: O=C(/C=C/c1cccc(C(F)(F)F)c1)N1CCCCC1=O] | 297.0977 | C₁₅H₁₄F₃NO₂ | 948 | 953.6 | 5.6 |
| SHJ-32 | 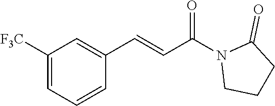 [SMILES: O=C(/C=C/c1cccc(C(F)(F)F)c1)N1CCCC1=O] | 283.082 | C₁₄H₁₂F₃NO₂ | 965 | 971.6 | 6.6 |
| SHJ-33 | 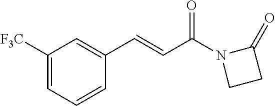 [SMILES: O=C(/C=C/c1cccc(C(F)(F)F)c1)N1CCC1=O] | 269.0664 | C₁₃H₁₀F₃NO₂ | 950.8 | 958.1 | 7.3 |
| SHJ-34 | 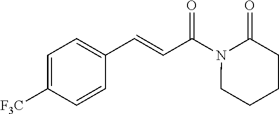 [SMILES: O=C(/C=C/c1ccc(C(F)(F)F)cc1)N1CCCCC1=O] | 297.0977 | C₁₅H₁₄F₃NO₂ | 950.8 | 955.6 | 4.8 |
| SHJ-35 | 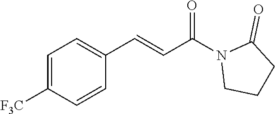 [SMILES: O=C(/C=C/c1ccc(C(F)(F)F)cc1)N1CCCC1=O] | 283.082 | C₁₄H₁₂F₃NO₂ | 982.8 | 989.3 | 6.5 |
| SHJ-36 | 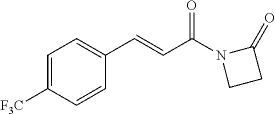 [SMILES: O=C(/C=C/c1ccc(C(F)(F)F)cc1)N1CCC1=O] | 269.0664 | C₁₃H₁₀F₃NO₂ | 970.7 | 979.4 | 8.7 |
| SHJ-38 | 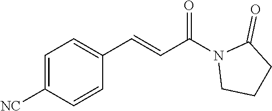 [SMILES: N#Cc1ccc(/C=C/C(=O)N2CCCC2=O)cc1] | 240.0899 | C₁₄H₁₂N₂O₂ | 970.7 | 975.7 | 5 |
| SHJ-39 | 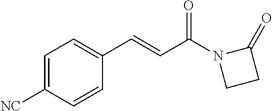 [SMILES: N#Cc1ccc(/C=C/C(=O)N2CCC2=O)cc1] | 226.0742 | C₁₃H₁₀N₂O₂ | 948 | 951.8 | 3.8 |
| SHJ-40 | 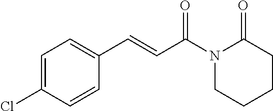 [SMILES: O=C(/C=C/c1ccc(Cl)cc1)N1CCCCC1=O] | 263.0713 | C₁₄H₁₄ClNO₂ | 991.5 | 998.8 | 7.3 |
| SHJ-41 | 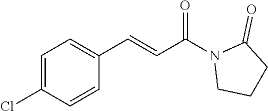 [SMILES: O=C(/C=C/c1ccc(Cl)cc1)N1CCCC1=O] | 249.0557 | C₁₃H₁₂ClNO₂ | 968.1 | 975.5 | 7.4 |
| SHJ-42 | 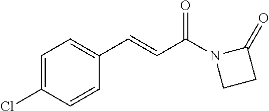 [SMILES: O=C(/C=C/c1ccc(Cl)cc1)N1CCC1=O] | 235.04 | C₁₂H₁₀ClNO₂ | 965.2 | 972 | 6.8 |
| SHJ-46 | 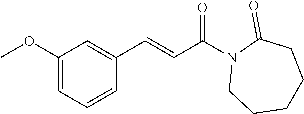 [SMILES: COc1cccc(/C=C/C(=O)N2CCCCCC2=O)c1] | 273.1365 | C₁₆H₁₉NO₃ | 942.6 | 955.1 | 12.5 |
| SHJ-48 | 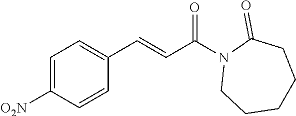 [SMILES: O=C(/C=C/c1ccc([N+](=O)[O-])cc1)N1CCCCCC1=O] | 288.111 | C₁₅H₁₆N₂O₄ | 949.8 | 953.8 | 4 |
| SHJ-49 | 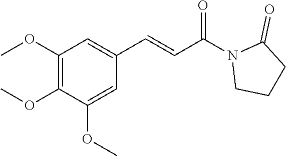 [SMILES: COc1cc(/C=C/C(=O)N2CCCC2=O)cc(OC)c1OC] | 305.1263 | C₁₆H₁₉NO₅ | 944.7 | 949.7 | 5 |
| SHJ-50 | 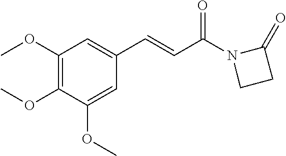 [SMILES: COc1cc(/C=C/C(=O)N2CCC2=O)cc(OC)c1OC] | 291.1107 | C₁₅H₁₇NO₅ | 993.9 | 1000.4 | 6.5 |
| SHJ-51 | 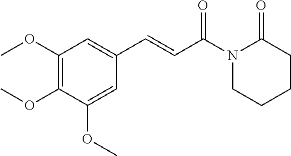 [SMILES: COc1cc(/C=C/C(=O)N2CCCCC2=O)cc(OC)c1OC] | 319.142 | C₁₇H₂₁NO₅ | 942.6 | 949.8 | 7.2 |
| SHJ-52 | 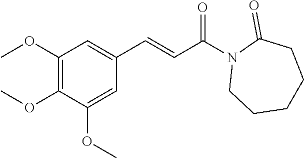 [SMILES: COc1cc(/C=C/C(=O)N2CCCCCC2=O)cc(OC)c1OC] | 333.1576 | C₁₈H₂₃NO₅ | 973.6 | 986 | 12.4 |
| SHJ-53 | 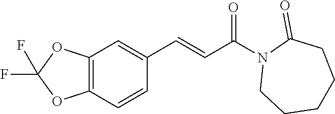 [SMILES: O=C(/C=C/c1ccc2c(c1)OC(F)(F)O2)N1CCCCCC1=O] | 323.0969 | C₁₆H₁₅F₂NO₄ | 948.2 | 957.1 | 8.9 |
| SHJ-54 | 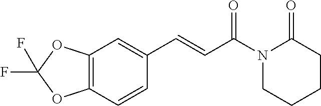 [SMILES: O=C(/C=C/c1ccc2c(c1)OC(F)(F)O2)N1CCCCC1=O] | 309.0813 | C₁₅H₁₃F₂NO₄ | 985.8 | 995.6 | 9.8 |
| SHJ-55 | 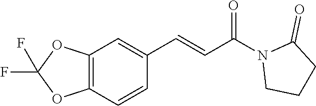 [SMILES: O=C(/C=C/c1ccc2c(c1)OC(F)(F)O2)N1CCCC1=O] | 295.0656 | C₁₄H₁₁F₂NO₄ | 960.2 | 966 | 5.8 |
| SHJ-56 | 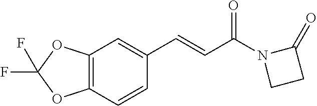 [SMILES: O=C(/C=C/c1ccc2c(c1)OC(F)(F)O2)N1CCC1=O] | 281.05 | C₁₃H₉F₂NO₄ | 950.8 | 959.8 | 9 |
| SHJ-57 | 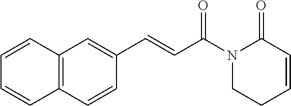 [SMILES: O=C1C=CCCN1C(=O)/C=C/c1ccc2ccccc2c1] | 277.1103 | C₁₈H₁₅NO₂ | 943.9 | 948.9 | 5 |

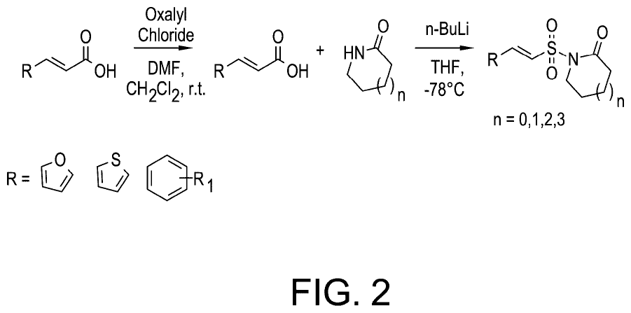

The piperlongumine derivatives of Table 1 may be synthesized by the compounds and formulas described in FIGS. 2-4A&B. The piperlongumine derivatives are derived from the following scheme of reactions:

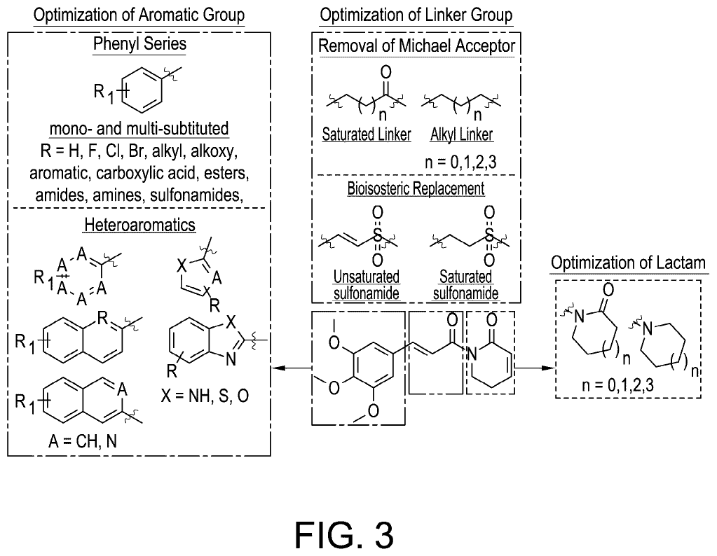

Now referring to FIG. 3, semi-synthetic optimization of natural products is a methodology that has led to FDA-approved drugs with significant clinical activity. To optimize PG therapeutic potential, we propose the following medicinal chemistry plan to further improve PG otoprotection and biopharmaceutical properties.

The analogs synthesized have focused on para-substituted aromatics to expand SAR on the aromatic ring, we propose two major aromatic series, i) mono- and multi-substituted phenyl rings and ii) unsubstituted and substituted heteroaromatics.

In Scheme 1, briefly, commercially available 3-aryl-α, β-unsaturated carboxylic acids were converted to acid chlorides with oxalyl chloride. Various lactams were activated with n-butyl lithium and were reacted with acid. 3-aryl-α, β-unsaturated carboxylic acids were converted to acid chlorides with oxalyl chlonde. Various lactams were activated with n-butyl lithium and were reacted with acid chlorides to yield final products. R1=3-methoxy, 3-trifluromethyl, 4-trifluromethyl 4-cyano, 4-nitro, 4-chloro, 3,4,5-trimethoxy 2,2-difluoroacetal (FIG 2).

In order to synthesize the piperlongumine derivatives with optimized aromatic groups i) mono-and multi-substituted phenyl rings, and il) unsubstituted and substituted heteroaromatics scheme 2 (FIG. 2) is followed:

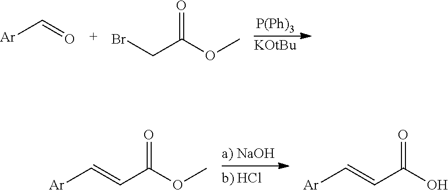

Scheme 2. Synthesis of β-aryl-α,β-unsaturated carboxylic acids.

the β-aryl-α, β-unsaturated carboxylic acids synthesized are followed by reactions of Scheme 1(FIG. 2).

In scheme 2, β-aryl-α, β-unsaturated carboxylic acids will be synthesized according to Scheme 2. Briefly, aryl or heteroaryl aldehydes will be reacted with methyl 2-bromo acetate under Wittig conditions. Soaponification of the methyl ester will yield β-aryl-α, β-unsaturated carboxylic acids. We plan to synthesize PG analogs with at least 50-75 unique aromatic groups.

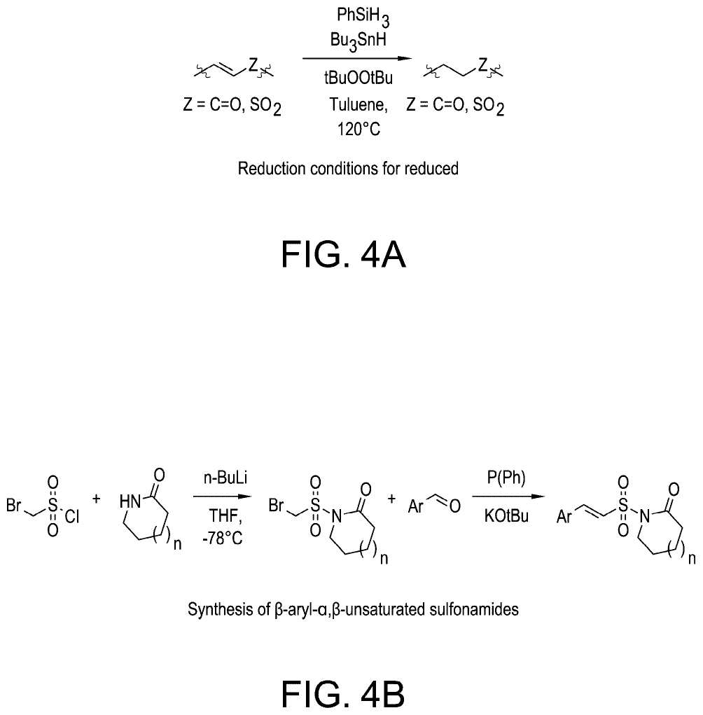

In order to synthesize the piperlongumine derivatives with optimized linker groups i) saturated amide linker, ii) reduced alkyl linker, iii) β-aryl-α, β-unsaturated sulfonamides, and iv) saturated sulfonamides scheme 3 (FIG. 4A) and 4 (FIG. 4B) are followed:

Saturated amide linker and alkyl linker groups result after Piperlongumine (E)-1-(3-(3,4,5-trimethoxyphenyl)acryloyl)-S,6-dihydropyridin-2(1H)-one analogs are synthesized according to Scheme 1 (FIG. 2) and reduced according to Scheme 3. β-aryl-α,β-unsaturated sulfonamides will be synthesized according to Scheme 4 (FIG. 4B). In scheme 4, 2-bromomethyl sulfonylchloride may be reacted with various lactams in the presence of n-butyl lithium, yielding the sulfonamide.

Compounds are revealed to protect against hair cell apoptosis. Compounds are identified as acting against hair cell loss in animals by the models and data presented. Models reveal properties necessary for an otoprotective compound such as high efficacy against hair cell loss, relatively low toxicity. Compounds are revealed to have high efficacy and high affinity in mouse and zebrafish models used to demonstrate protection against hair cell loss. The lateral-line neuromasts of zebrafish are a valuable model for testing compounds protective against hearing loss in vivo, as their HCs are considered homologous to those in the mammalian inner ear and are readily accessible to drugs in vivo. Teitz et al., J. Exp. Med. 2; 215 (4):1187-1203 (2018). Mouse models involving embryonic fibroblast cell viability have shown effective in validating therapeutic uses of compounds against hearing loss due to cancer treatment such as cisplatin, noise, antibiotics, and aging. Teitz et al., J. Exp. Med. 2; 215 (4):1187-1203 (2018).

The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration. Compounds can be synthesized by a variety of methods known in the art. The nature of the pharmaceutical compositions for administration is dependent on the mode of administration and can readily be determined by one of ordinary skills in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

The preferred method of delivery is systemic administration. The term “systemic administration” includes oral or parenteral administration Parenteral administration delivers the compound into the body by means that bypass the GI tract. These methods include a variety of injections, as well as topical, transmucosal, and pulmonary routes. The term “parenteral administration” includes an intravenous injection. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm. Intravenous administration is the best way to deliver a precise dose quickly and in a well-controlled manner throughout the body.

A cyclodextrin derivative such as hydroxypropyl-β-cyclodextrin (HP-β-CD) could be used as a solubilizing agent for piperlongumine or a derivative thereof for Intravenous administration. An exemplary 20% (w/v) hydroxypropyl-β-cyclodextrin solution can be used to increase the solubility of piperlongumine or a derivative thereof for intravenous administration. See, e.g. V. Aiassa et al. “Cyclodextrins and Their Derivatives as Drug Stability Modifiers” Pharmaceuticals (Basel). 2023 August; 16 (8): 1074.

In various aspects, the disclosed pharmaceutical compositions include the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. Pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds. The pharmaceutical carrier employed can be, for example, a solid, liquid, or gas.

The pharmaceutical compositions of the present invention include the compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. Pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability.

In various aspects, the compounds of this invention can be used in combination with one or more other drugs, in the form of a kit, to prevention, control, amelioration, or reduction of risk of hearing impairments, when the other drugs can have been known to impair hearing such as an antibiotic. Certain antibiotics, especially aminoglycosides (such as gentamicin, streptomycin, and neomycin) Hearing-related side effects from these antibiotics are most common in people who have kidney disease or who already have ear or hearing problems.

### EXAMPLE 1

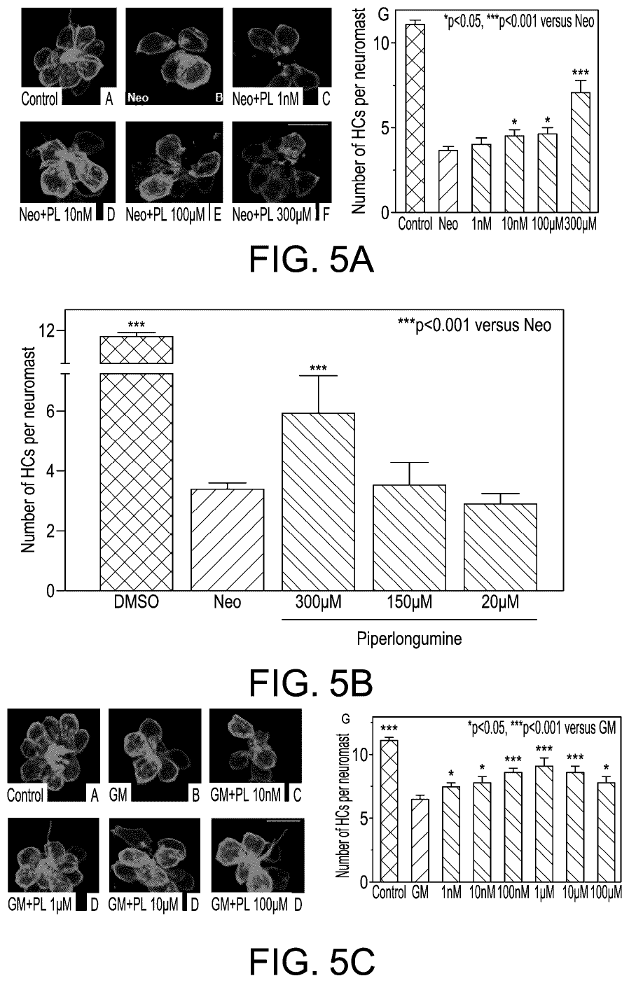

Now referring to FIGS. 5A-C, (E)-1-(3-(3,4,5-trimethoxyphenyl)acryloyl)-S,6-dihydropyridin-2(1H)-one) protects from aminoglycoside induced hair cell loss in zebrafish lateral line neuromasts in vivo. Zebrafish lateral line neuromast HC counts are commonly used in vivo models for screening otoprotectants. Danio rerio experimental larvae were obtained by pair mating of adult fish maintained at Creighton University by standard methods approved by the Institutional Animal Care and Use Committee. Fish used were Tg(pou4f3:mGFP) expressing a membrane bound GFP in HCs. Experimental fish were maintained at 28.5° C. in E3 media (5 mM NaCl, 0.17 mM KCl, 0.33 mM CaCl2 and 0.33 nM MgSO4, pH 7.2). Animals were cryoanaesthetized after drug treatment and prior to fixation. The neuromasts inspected, SO3 and O1-2, were part of the cranial system and included the otic, middle, and opercular neuromasts. The lateral-line neuromasts of zebrafish are a valuable system for testing protectivity of compounds against aminoglycoside toxicity in vivo, as their HCs are considered homologous to those in the mammalian inner ear and are readily accessible to drugs.

For the screenings, 5-day post-fertilization (dpf) Tg(brn3c:GFP) larvae

were pre-incubated with (E)-1-(3-(3,4,5-trimethoxyphenyl)acryloyl)-S,6-dihydropyridin-2(1H)-one at 10 nM, 10 nM, 1 μM, 10 μM, 100 μM and 300 μM for 1 hour followed by co-incubation with either 200 μM Neomycin (Neo) for 30 minutes (as shown in FIGS. 5A and B), or 100 μM gentamycin (GM) for 1 hour (as shown in FIG. 5C). DMSO, Neo alone, and GM alone were used as a control for their respective experiments (FIGS. 5A and C).

Subsequently, animals were transferred to E3 water for 5 hours and fixed in 4% paraformaldehyde (PFA) overnight. Neuromast HCs were immunolabeled with anti-otoferlin (HCS-1, DSHB) and anti-GFP (NB100-1614, Novus Biologicals). These two markers were used to detect and count neuromast HCs to reduce the chances of missing some of the HCs after the treatment since as it was previously noticed that incubation with the compounds can affect GFP expression more difficult to detect under a fluorescence microscope. Otic, middle, and opercular neuromasts were identified, and HCs at SO3 (supraorbital line neuromast) and O1-2 (Otic line neuromasts) were manually counted using a Zeiss AxioSkop 2 fluorescence microscope with a 40× oil objective. Control animals were treated with DMSO (−) or Neo or GM alone (+) Animals were then fixed and immunostained for GFP and otoferlin. Quantification was done by assessing three neuromasts in the same anatomic location of each animal (n=5). The data is plotted as mean+SD. Statistical testing was done with One-way ANOVA and Dunnett post-hoc test (*P<0.05, **P<0.01, ***P<0.001 compared to either Neo or GM, respectively).

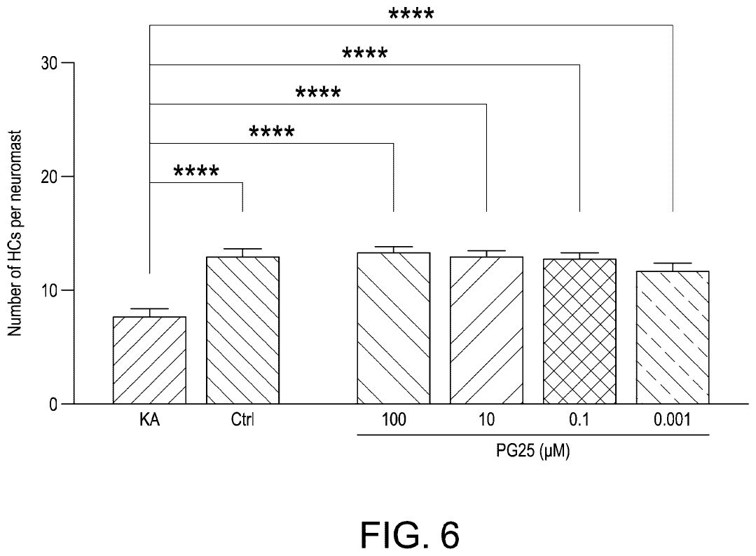

Now referring to FIG. 6, piperlongumine-25 protects neuromast hair cells from excitotoxic damage induced by kainic acid (KA). For the screening of piperlongumine derivatives for protection against noise-induced hearing loss we employed a zebrafish model that mimics excitotoxic damage. Five-day post-fertilization (dpf) Tg(brn3c:GFP) larvae were incubated with 300 μM of KA for 1 hour followed by piperlongumine-25 for 2 hours at 0.001 μM, 0.1 μM, 10 μM, and 100 μM. DMSO and KA were used as controls. Additionally, 5-day post-fertilization (dpf) Tg(brn3c:GFP) larvae were incubated with piperlongumine-25 alone for 2 hours to confirm that by itself does not result in any general toxicity.

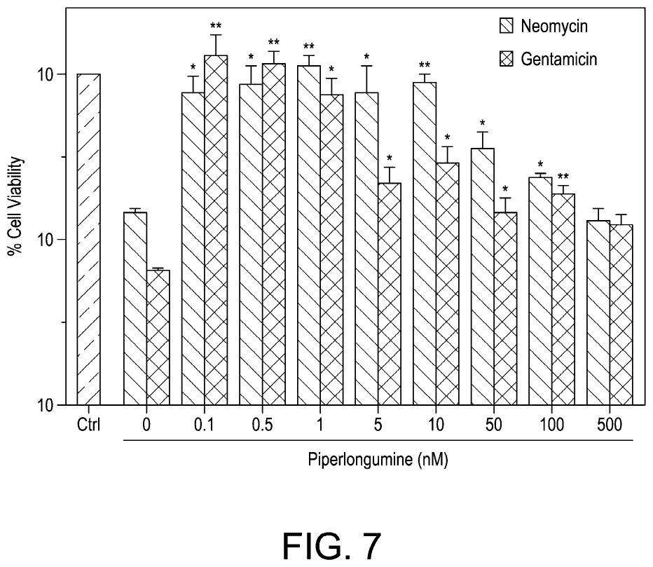

Now referring to FIG. 7 (E)-1-(3-(3,4,5-trimethoxyphenyl)acryloyl)-S,6-dihydropyridin-2(1H)-one protects mouse embryonic fibroblast (MEFs) against aminoglycosides. MEFs were incubated for 15 hours with neomycin (100 μM) or gentamicin (8 μM) with or without (E)-1-(3-(3,4,5-trimethoxyphenyl)acryloyl)-S,6-dihydropyridin-2(1H)-one (0.1 nM-5 μM) at 0.1 nM, 0.5 nM, 1 nM, 5 nM, 10 nM, 50 nM, 100 nM, and 500 nM. Cell viability was assessed employing MTT (3-(4-,5-dimethylthiazol-2-yl)-2,5-diphenyltetrazolium bromide) viability kit test (Thermo Fisher). The data is plotted as mean+SD. Statistical testing was done with One-way ANOVA and Dunnett post-hoc test (*P<0.05, **P<0.01 compared to either Neo or GM, respectively). 100% protection by (E)-1-(3-(3,4,5-trimethoxyphenyl)acryloyl)-S,6-dihydropyridin-2(1H)-one was observed at 0.1 nM to 10 nM against neomycin and 0.1 nM to 1 nM against gentamycin.

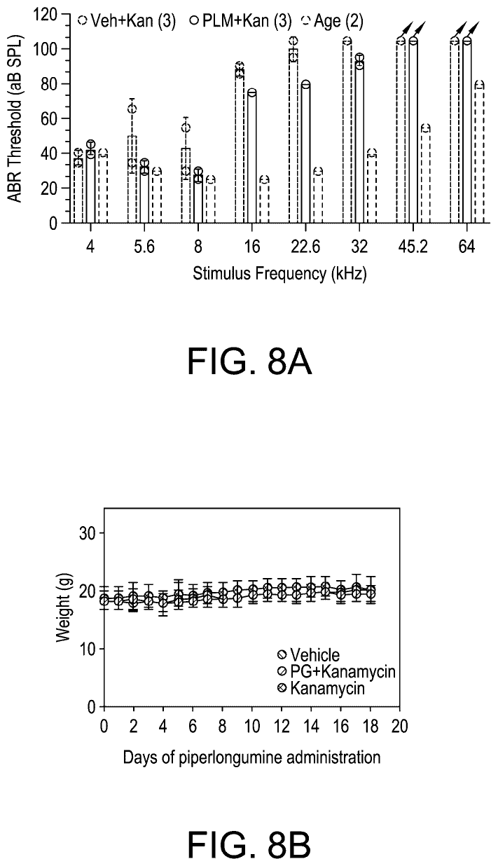

Now referring to FIG. 8A, protects piperlongumine from aminoglycoside toxicity in mice in vivo. 6-7-week-old C57BL/6 (Cdh23 corrected) were used, with a mix of males and females across experiments. Procedures were approved by the IACUC committee of the Creighton University.) Mice were treated with piperlongumine (40 mg/kg b.w., for 17 days, IP) with or without kanamycin treatment (700 mg/kg b.w. for 14 days twice a day, s.q.). piperlongumine's protection against hearing impairment loss was assessed by measuring ABR threshold shifts in the mouse ear.

Animals were anesthetized with a mixture of ketamine/xylazine before ABR assessment. Subcutaneous needle electrodes were inserted behind the pinna (inverting), vertex of the skull (non-inverting) and base of the tail (ground). Tone bursts of 5 ms duration with 0.5 ms cosine-squared envelopes delivered at a rate of 21 stimuli per second with alternating polarity were generated using BioSigRZ software and RZ6 multi I/O processor system (Tucker-Davis Technologies, FL). Stimuli were presented as open field via a speaker (MF1; TDT, FL) placed 10 cm in front of the pinna of the animal. Evoked responses were amplified (20×), bandpass filtered (300-3,000 Hz) and an average of 512 responses of 10 ms duration was recorded. Stimulus intensity was decreased in 5 dB increments, starting from 100 dB SPL to 0 dB SPL. Thresholds at 4, 5.6, 8,16, 22.6, 32, 45.2, and 64 kHz were identified by visual inspection from stacked waveforms as the lowest level at which reproducible response could be identified. Before the start of every session, stimulus presenting speaker (MF1) was calibrated with a ¼″ microphone (PCB-378C10; PCB Piezotronics, NY) that was also placed 10 cm in front of the speaker. Similar experiments have been previously described in Rai V. et al., Sci Rep. 2020 Sep. 16; 10 (1): 15167.

Age-control group mice were administered saline. Significant differences in threshold were observed at 22.6 kHz (p=0.0408, 2-way ANOVA with Holm Sidak's multiple comparison). Data are presented as mean±SD, n=3-4/group. Now referring to FIG. 8B, kanamycin alone and in combination with (E)-1-(3-(3,4,5-trimethoxyphenyl)acryloyl)-S,6-dihydropyridin-2(1H)-one showed no general toxicity in C57BL/6 mice (Cdh23 corrected) during the 17-day exposure period.

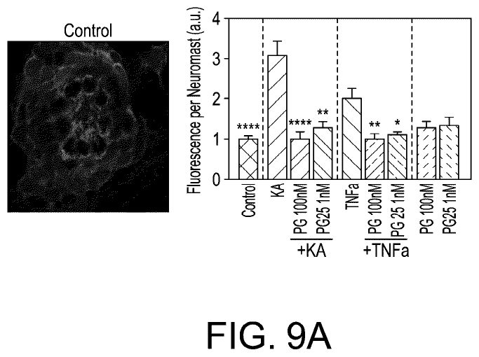

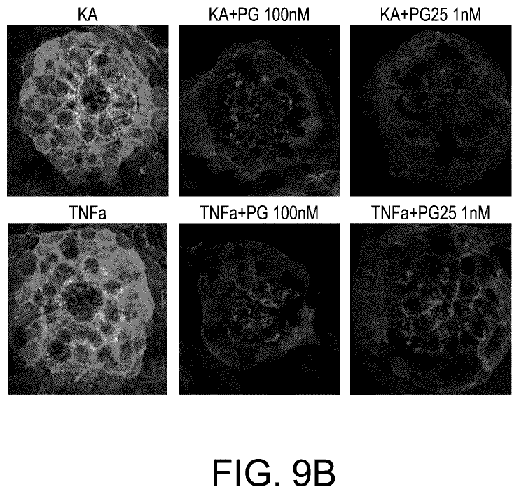

Now referring to FIG. 9, to confirm that (E)-1-(3-(3,4,5-trimethoxyphenyl)acryloyl)-S,6-dihydropyridin-2(1H)-one and its derivates work by inhibiting NF-kB activity a set of experiments were performed. Zebrafish reporter line that expresses GFP under the NF-kB promoter were used. In individual experiments, at 5 dpf animals were incubated with vehicle (E3 water), KA 300 μM for 1 hour or 10 ng/mL TNFa for 30 minutes to induce the NF-kB pathway. Next, zebrafish were incubated with vehicle (DMSO 0.1%), or with (E)-1-(3-(3,4,5-trimethoxyphenyl)acryloyl)-S,6-dihydropyridin-2(1H)-one or one of its derivatives at 1 nM and 100 nM for 2 hours. Zebrafish neuromast were fixed and immunostained for GFP (green) and otoferlin (red). The fluorescence intensity of GFP (used as a proxy for NF-kB activation) was quantified employing ImageJ and expressed as arbitrary florescence units per neuromast. TNFa, an activator of NF-kB, was used as a positive control. Zebrafish incubated with (E)-1-(3-(3,4,5-trimethoxyphenyl)acryloyl)-S,6-dihydropyridin-2(1H)-one at 100 nM or piperlongumine-25 (PG25 ((SHJ-25)) at 1 nM showed a reduction of NF-kB pathway activation compared to fish exposed to KA or TNFa alone. Piperlongumine (E)-1-(3-(3,4,5-trimethoxyphenyl)acryloyl)-S,6-dihydropyridin-2(1H)-one at 100 nM or piperlongumine-25 PG25 ((SHJ-25)) at 1 nM showed a reduction of NF-kB pathway activation compared to fish exposed to KA or TNFa alone.

Zebrafish were incubated with vehicle (control), KA 300 μM (KA), or TNFa (10ng/ml) (TNFa) alone or in combination with Piperlongumine (E)-1-(3-(3,4,5-trimethoxyphenyl)acryloyl)-S,6-dihydropyridin-2(1H)-one or one of its derivatives for 2 hours at concentrations of 1 nM or 100 nM. Animals were fixed and immunostained for GFP (green) and otoferlin (red). GFP fluorescence intensity was quantified with ImageJ and expressed as mean+/−SEM. Statistical analysis: One-Way ANOVA. (*p,0.05, **p<0.01, ****p<0.0001, compared to the corresponding ototoxin alone). Piperlongumine (E)-1-(3-(3,4,5-trimethoxyphenyl)acryloyl)-S,6-dihydropyridin-2(1H)-one 100 nM or Piperlongumine-25 (PG25 1nM showed a reduction of NF-kB pathway activation compared to fish exposed to KA or TNFa alone.

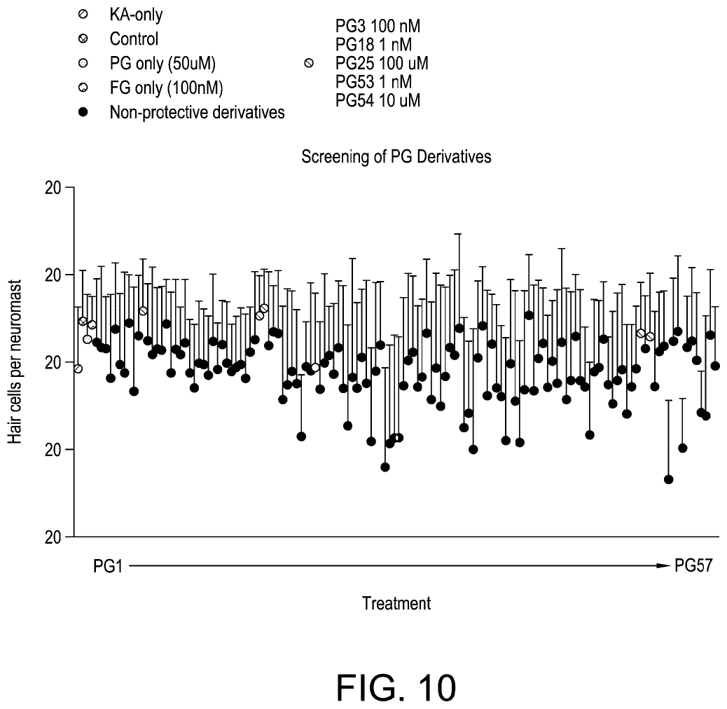

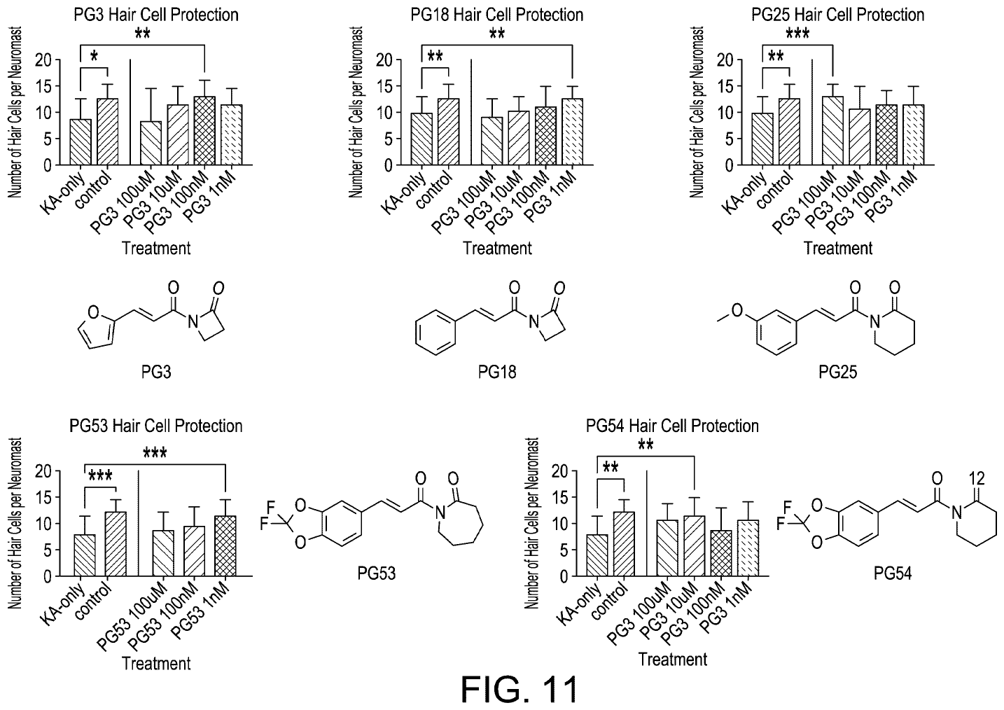

Now referring to FIGS. 10 and 11 Of the derivates of Piperlongumine tested, PG3 (SHJ-3), PG 18 (SHJ-18), PG25 ((SHJ-25), PG 53 (SHJ-53) and PG 54 (SHJ-54) had better performance than Piperlongumine against kainic acid (KA) induced hair cell death.

FIG. 10 shows piperlongumine derivatives were tested in a zebrafish model for excitotoxicity. 5 dpf fish were incubated with KA 300 μM for 1 hour followed by a two-hour incubation with one of the piperlongumine derivatives at 1nM to 100 μM. Animals were fixed and immunostained for the hair cell marker, otoferlin. Neuromast hair cells were counted under a fluorescence microscope. At least 3 neuromast of the rostral lateral line were inspected per fish, in a total of 6 fish per treatment. Results are presented as mean+/−SD. Control, KA only, PG only, PG+KA, derivatives that were not protective against excitotoxicity, derivatives that performed better than PG. (The legend shown in the FIG. 10),

FIG. 11 shows PG3, PG18, PG25, PG53 and PG54 performed better than piperlongumine in a zebrafish model for excitotoxicity. 5 dpf fish were incubated with KA 300 μM for 1 hour followed by one of the PG derivatives at different concentrations for additional 2 hours. Animals were fixed and immunostained for otoferlin. Neuromast hair cells were quantified in at least 3 rostral neuromasts per fish in a total of 6 fish per treatment. Results are expressed as mean+/−SD. Statistical analysis: One-way ANOVA *P<0.05, **P<0.01, P<0.001 versus KA alone. These five piperlongumine derivatives performed better than piperlongumine in at least one dose.

### EXAMPLE 2

### Materials And Methods

Animal care. Zebrafish and mice were maintained at Creighton University Animal Resource Facilities by standard methods and per the Institutional Animal Care and Use Committee. For piperlongumine ototoxicity and FM1-43 uptake experiments, we used 5-6 dpf wild type (TuAB) and Tg(brn3c:GFP) zebrafish lines. For proximal tubule integrity assay, 5-6 dpf Tg(Ifabp::1/2vdbpm-Cherry) fish was used. Z. Chen, A. Luciani, J. Mateos, G. Barmettler, R. Giles, S. Neuhauss, O. Devuyst, Transgenic Zebrafish Modeling Low-Molecular-Weight Proteinuria and Lysosomal Storage Diseases. Kidney Int. 97, 1150-1163 (2020) Fish were maintained at 28.5° C. in E3 media with a 14-hour light/10-hour dark cycle. The B6N (Cg)-Cdh23^{tm2.1/kjn} mouse line was used for the kanamycin chronic studies and was maintained with a 12-hour light/dark cycle and free access to food and water.

Zebrafish experiments Ototoxicity studies: Dose-response curves were obtained employing various piperlongumine concentrations (Cayman Laboratories) (8 μM to 8 pM in half steps) in DMSO. After one hour, fish were co-incubated with piperlongumine and 400 μM of kanamycin (VWR) for 5 hours, and HCs manually counted using a Zeiss AxioSkop2 fluorescence microscope. The neuromasts inspected were part of the cranial system and included the otic, middle, and opercular neuromasts. See M. Zallocchi, S. Hati, Z. Xu, W. Hausman, H. Liu, D. He, J. Zuo, Characterization of Quinoxaline Derivatives for Protection against latrogenically-Induced Hearing Loss. JCI Insight 6, 141561 (2021).FM1-43 uptake: Dye uptake was used as a proxy for mechanotransduction channel activity. Experiments were performed in the presence of piperlongumine 150 pM (EC₅₀) or 15 nM (×100 EC₅₀).O. Ogun, M. Zallocchi, Clarin-1 Acts as a Modulator of Mechanotransduction Activity and Presynaptic Ribbon Assembly. J. Cell. Biol. 207, 375-391 (2014).

Kidney proximal tubule activity: The Tg(Ifabp::1/2vdbpm-Cherry) zebrafish line was used for these experiments. Animals were incubated with kanamycin in the presence of piperlongumine (PL3˜150 pM [EC₅₀], PL2=150 nM and PL1=8 μM).Z. Chen, A. Luciani, J. Mateos, G. Barmettler, R. Giles, S. Neuhauss, O. Devuyst, Transgenic Zebrafish Modeling Low-Molecular-Weight Proteinuria and Lysosomal Storage Diseases. Kidney Int. 97, 1150-1163 (2020).

Cell-based, CYP450 and Disc diffusion assays. Viability assay: HEI-OC1 cells were grown, and the cell viability was assessed with PRESTOBLUE Cell Viability Reagent (ThermoFisher Scientific). See G. Kalinec, P. Thein, C. Park, F. Kalinec, HEI-OC1 Cells as a Model for Investigating Drug-Cytotoxicity. Hear. Res.335, 105-117 (2016).

Colchicine incubations: HEI-OC1 cells were incubated for 8 hours with 25 μM of piperlongumine or 10 μM colchicine. Fixed cells were immunostained for a-tubulin.

CYP450 assay: Cytochrome activity in the presence of piperlongumine was measured with the VIVID CYP3A5 Green Screening Kit (ThermoFisher). Piperlongumine was used at 1 mM, 0.2 mM, 0.04 mM (concentrations in the plateau region of the dose-response curve), 8 nM, 1.6 nM (concentrations in the upper part of the linear function), and 0.32 nM (concentration in the lower part of the linear dose-response function). Ketoconozale, 10 mM was used as a positive control for inhibition of CYP450 activity.

An evaluation of aminoglycoside efficacy in the presence of piperlongumine was performed. See M. Zallocchi, S. Hati, Z. Xu, W. Hausman, H. Liu, D. He, J. Zuo, Characterization of Quinoxaline Derivatives for Protection against latrogenically-Induced Hearing Loss. JCI Insight 6, 141561 (2021).

Mouse treatment. The paradigm to generate the kanamycin ototoxicity model in mice is as described below. See M. Jiang, H. Li, A. Johnson, T. Karasawa, Y. Zhang, W. Meier, F. Taghizadeh, A. Kachelmeier, P. Steyger, Inflammation Up-Regulates Cochlear Expression of TRPV1 to Potentiate Drug-Induced-Hearing Loss. Sci. Adv. 5, eaaw1836 (2019).Seven-to-eight weeks old B6N (Cg)-Cdh23^{tm2.1/kjn} mice received vehicle (corn oil) or piperlongumine 40 mg/kg b.w. in corn oil intraperitoneally (IP) for 17 days. Kanamycin or vehicle (saline solution) administration was given subcutaneously twice a day for 14 days, starting one day after the first piperlongumine treatment. Weights were monitored and the piperlongumine and kanamycin doses were adjusted every 5 days to reflect the changes in total body weight. Animals were sacrificed 4 weeks after the last dose and the post-treatment hearing tests.

Hearing tests, The ABR acquisition and data management were performed using a Tucker-Davis Technologies (TDT, Alachua, FL) RZ6 Multi I/O processor and BioSigRZ software. Stimuli were presented by a TDT MF1 driver placed 10 cm from the left ear of each animal. Tone burst stimuli of 5 ms duration at half-octave frequency intervals from 4.0-64 kHz were presented at 19/s. At each frequency, an intensity series was presented from 20-100 db SPL in 5 dB steps. For DPOAEs the primary tones f1 and f2 were generated and shaped using TDT's hardware. DPOAEs were recorded in the form of level/frequency functions; f2/f1 was fixed at 1.2, with the level of the f2 (L2) 10 dB lower than the f1 level (L1), signal was collected by an ER10B+(Etymotic) microphone.

Endocochlear potentials. Endocochlear potentials were measured at least 24 hours after the last hearing test and the inner ears were immediately isolated and processed for immunohistochemistry. For the endocochlear potentials, a round window approach was taken from the cochlear basal turn. See R. Hilhorst, L. Houkes, A. van den Berg, R. Ruijtenbeek, Peptide Microarrays for Detailed, High-Throughput Substrate Identification, Kinetic Characterization, and Inhibition Studies on Protein Kinase A. Anal. Biochem. 387, 150-161 (2009).

Animals received piperlongumine (40 mg/kg) or corn oil IP. After two hours, the inner ears were microdissected and homogenized, and piperlongumine extracted and processed for LC-MS/MS. J. Liu, W. Liu, Y. Lu, H. Tian, C. Duan, L. Lu, G. Gao, X. Wu, X. Wang, H. Yang, Piperlongumine Restores the Balance of Autophagy and Apoptosis by Increasing BCL2 Phosphorylation in Rotenone-Induced Parkinson Disease Models. Autophagy 14, 845-861 (2018).

Kinome analyses. Identification of significant differential AKT family kinase activity: Twenty-three AKT kinase family putative target peptides were identified by the kinome random sampling analyzer (KRSA) package. X. Wang, Y. Bian, C. Wong, J. Lu, S. Lee, TRPV1 Modulator Ameliorates Alzheimer-Like Amyloid-β-Neuropathology via Akt/Gsk38-Mediated Nrf2 Activation in the Neuro-2a/APP Cell Model. Oxi.Med. Cell. Long. e1544244 (2022). Log2-fold changes in peptide activity were calculated by comparing kanamycin, PL, or kanamycin+PL to vehicle control. For each peptide, an average of triplicates was used per condition. The technical triplicates of each condition were highly reproducible. Two-way ANOVA was used to identify significant (***P<0.001).Methods Omnibus for the PamGene Kinome Array: The STK PamGene12 chips have 144 reporter peptides. G. Manning, Genomic Overview of Protein Kinases. WormBook 1-19 (2005). The sample is added to one of the 4-well peptide chip and phosphorylation is detected in real-time with fluorescent antibodies. Fluorescent intensity is used as a proxy for the extent of reported peptide phosphorylation and altered kinase activity can be directly measured. Of the ˜500 kinases in the human genome, 65% can be mapped to the STK chip with a detection sensitivity in the picogram range.

Kinome Array Protocols. Samples were prepared according to PamGene Corp (https://pamgene.com/ps12/). Peptide phosphorylation was monitored during the incubation. The primary output is images that are then pre-processed to quantify the activity at each peptide level using the PamGene's BioNavigator software (https://pamgene.com/wp-content/uploads/2020/09/BioNavigator-User-Manual-vs2.3-2020.pdf), inactive peptides (raw signal <=5) are removed from the analysis. The dynamic range of the raw signal intensities is typically 0-3,000. The linear regression slope of the signal intensity as a function of exposure time is used to represent the peptide phosphorylation intensity for downstream comparative analyses. The signal ratio between case and control samples is used to calculate fold change (FC) values. Peptides with a FC of at least 15% (FC>1.15 or FC<0.85) are considered differentially phosphorylated. This threshold was chosen based on previous reports that suggest minor changes in kinase activity are sufficient to trigger biologically relevant changes (53). Peptides that have a very low signal or an R²<0.90 are excluded from subsequent analyses. Peptides spotted on the array can be phosphorylated by more than one kinase. Four different software packages are deployed for the assignment of upstream kinases: Upstream Kinase Analysis (UKA) package (Pamgene Corps'-Hertengobosch, Netherlands) relies on a curated database of kinase-substrate interactions created by the PamGene Corp. KRSA, relies on a curated database of upstream kinases that were identified using GPS 3.0 and Kinexus Phosphone. M. Zallocchi, D. Meehan, D. Delimont, J. Rutledge, M. Gratton, J. Flannery, D. Cosgrove, Role for a Novel Usher Protein Complex in Hair Cell Synaptic Maturation. PLOS One 7, e30573 (2012).

Integration of Upstream Kinase Assignments Across Packages. All the tools above utilize different methods to assign upstream kinases. To identify consensus upstream kinases across datasets, we utilize the software Creedenzymatic that takes in the results from the analyses and generates a consensus figure with kinases deconvolved and ranked based on their presence in the results. M. Zallocchi, D. Meehan, D. Delimont, J. Rutledge, M. Gratton, J. Flannery, D. Cosgrove, Role for a Novel Usher Protein Complex in Hair Cell Synaptic Maturation. PLOS One 7, e30573 (2012).

Immunoblot assay. For the immunoblot studies, organs of Corti were collected from the different treatments, processed, and used to run western blots for AKT1 and ERK proteins. See K. Kane, C. Longo-Guess, L. Gagnon, D. Ding, R. Salvi, K. Johnson, Genetic Background Effects on Age-Related-Hearing-Loss Associated with Cdh23 Variants in Mice. Hear. Res. 283, 80-88 (2012).

Immunohistochemistry and confocal microscopy, Fish were fixed and immunostained for the HC marker, otoferlin (HC count), or directly washed and mounted (FM1-143 uptake). See K. Kane, C. Longo-Guess, L. Gagnon, D. Ding, R. Salvi, K. Johnson, Genetic Background Effects on Age-Related-Hearing-Loss Associated with Cdh23 Variants in Mice. Hear. Res. 283, 80-88 (2012). The inner ears were microdissected and processed as previously described (54). Samples were immunostained for myosin7A, Ctbp2, Sox2, neurofilament H, pAKT1, and TRPV1. Images were captured in a Zeiss LSM 700 at room temperature with automatically set sectioning and processed with ZEN black edition software. Z-stacks are presented as flat Z-projections. Total fluorescence intensities were calculated using ImageJ (NIH). The neuronal fiber analysis was performed using the filament tracing tool from Imaris. The OHCs were manually counted. Ctbp2 puncta was quantified using the “Analyzed Particles” tool in ImageJ previous thresholding. Adjusted parameters were pixel size=0.2-infinity, circularity 0.50-1.00. Results were visually confirmed for any missing or additional puncta.Molecular docking and MD simulation. The receptors and ligands preparation were performed using the protein wizard tool from Maestro. To place piperlongumine in the same allosteric binding pocket presented in the crystal structure of TRVP2 (PDB code: 6WKN), we overlaid this protein with the TRVP1 (PDB code: 7LPE) homolog using the MatchMaker structure comparison tool. See O. Trott, A. Olson, AutoDock Vina: Improving the Speed and Accuracy of Docking with a New Scoring Function, Efficient Optimization, and Multithreading. J. Comput. Chem. 31, 455-461 (2010). Following, we selected the corresponding binding residues in TRVP1 to identify the binding pocket, Next, using Autodock Vina PL was docked, and the most stable conformation generated (best binding affinity) was selected to be analyzed by MD simulation. Autodock Vina uses a fixed target/flexible ligand approach, with a generic algorithm for the search step, and a hybrid function based on empirical data combined with a knowledge-based function, In the molecular docking step, the center of the selected residues was used as the reference to create the cubic virtual space (grid) needed to perform the search. The grid center was set at 34.5 Å (x), 32.4 Å (y) and 25.1 Å (z). This positioning and volume were adequate to cover the binding site. The exhaustiveness parameter (which indicates the time spent searching for a global minimum in each docking calculation) was set to 60. This way, the chances of obtaining both a suitable molecular docking methodology and adequate and reliable best poses for the purpose of the study are increased. MD simulations were used to analyze the dynamic behavior of the TRPV1 receptor bound to piperlongumine, agonist control (CP, PDB code:7LPE) or the apo-receptor.E. Pettersen, T. Goddard, C. Huang, G. Couch, D. Greenblatt, E. Meng, T. Ferrin, UCSF Chimera-a Visualization System for Exploratory Research and Analysis. J. Comput. Chem. 2, 1605-1612 (2004). All simulations were conducted using rat TRPV1 (PDB code:7LPE). A phosphatidylcholine (POPC) membrane was placed on the receptor according to the orientation calculated using the online Orientation of Proteins in Membrane Server-OPM. Each receptor-ligand complex or apo-receptor was placed in an orthorhombic box with periodic boundaries. The box was filled with TIP3P water and Na⁺ or CI⁻ ions, which were added to neutralize the system. In addition, 0.15M NaCl was added to mimic physiological ion concentration. Next, the system was relaxed using positionally restricted dynamics following the standard Desmond protocol K. Roos, C. Wu, W. Damm, M. Reboul, J. Stevenson, C. Lu, M. Dahlgren, S. Mondal, W. Chen, L. Wang, R. Abel, R. Friesner, E. Harder, OPLS3e: Extending Force Field Coverage for Drug-Like Small Molecules. J Chem. Theory Comput. 15, 1863-1874 (2019). All simulations were performed on an NPT (constant particle number, pressure, and temperature) ensemble at a temperature of 300K and pressure of 1,013 bar. The MD simulation time was set to 250 nanoseconds for all complexes. The energy (kcal.mol⁻¹) was recorded at each 1.2 picosecond interval. All simulations were performed with Desmond GPU using the OPLS3e force field through the Holland Computer Center. The OPLS3e force field is based on optimized potentials for liquid simulations (OPLS). The post-MD analyses were performed using the simulation interaction diagram tool and images were created using Maestro graphical interface E. Pettersen, T. Goddard, C. Huang, G. Couch, D. Greenblatt, E. Meng, T. Ferrin, UCSF Chimera-a Visualization System for Exploratory Research and Analysis. J. Comput. Chem. 2, 1605-1612 (2004).

KPT2 cell experiments. Experiments were performed as follows. Briefly, for AKT activity assessment, KPT2-cells were incubated for 5 minutes with DMEM+1.8 mM CaCl₂ and CP 3μM, CP+PL (1 μM or 0.1μM) or PL-only (1μM), following by immunoblot or immunocytochemistry processing. For GTTR uptake experiments, KPT2-cells were incubated for 3 minutes with DMEM+1.8 mM CaCl₂+5 μg/ml GTTR alone or CP 3 □M, capsazepine 15 □M or CP+PL 1 μM following by confocal processing. D. Mukherjea, S. Jajoo, K. Sheehan, T. Kaur, S. Sheth, J. Bunch, C. Perro, L.P. Rybak, V. Ramkumar, NOX3 NADPH Oxidase Couples Transient Receptor Potential Vanilloid 1 to Signal Transducer and Activator of Transcription 1-Mediated Inflammation and Hearing Loss. Antioxid. Redox Signal. 14, 999-1010 (2011).

Antibodies and special reagents.Antibodies used: anti-otoferlin (HCS-1, DSHB) 1:100, anti-myosin7A (Proteus Biosciences) 1:400, anti-Ctbp2 (BD Biosciences) 1:200, anti-neurofilament H (Millipore) 1:1,000, anti-μ tubulin (Sigma- Aldrich) 1:200, anti-TRPV1 (ABclonal) 1:200, anti-AKT1Ser473, anti-panAKT, anti-ERK1/2T202/Y204 and anti-ERK1/2 (CST) 1:750, anti-Sox2 (ThermoFisher) 1:300.

piperlongumine was obtained from Cayman Chemicals (#11006), kanamycin for zebrafish (VWR, #75856-686), kanamycin for mice (Sigma-Aldrich, #K0254).

Study Design and Statistical Analysis. The objective of this study was to test whether piperlongumine can preserve hearing function during aminoglycoside treatment. The sample size was calculated based on previous hearing tests performed in our laboratory. Only one animal (female) was excluded during the hearing test experiments since its ABR baselines were already abnormal. After the hearing baseline measurements, animals were allowed to recover from anesthesia for 1-3 days before the initiation of the treatment. Mice were randomized into the four groups with an equal number of females and males and the treatment was performed for a total of 17 days (considering piperlongumine administration). Each mice received an alphanumerical code associated with the treatment to which the person(s) conducting the hearing tests and analysis was blinded. After 4 weeks of post-treatment the inner ears were microdissected. For in vivo studies, each animal (fish/mouse) within a group was considered as independent biological replicates. For the cell studies, experiments were performed three times, and each was considered an independent biological replicate.Brown-Forsythe-Welch ANOVA followed by Dunnett's T3 post-test for multiple comparisons, one-way or two-way ANOVAs followed by Dunnett's post-test for multiple comparisons, were performed using GraphPad v10.0.2. Results were expressed as mean+SEM. 5-6 fish were used per experiment, and at least 3 rostral neuromasts were inspected. Mouse numbers were included in the figures (between brackets) or represented as circles.

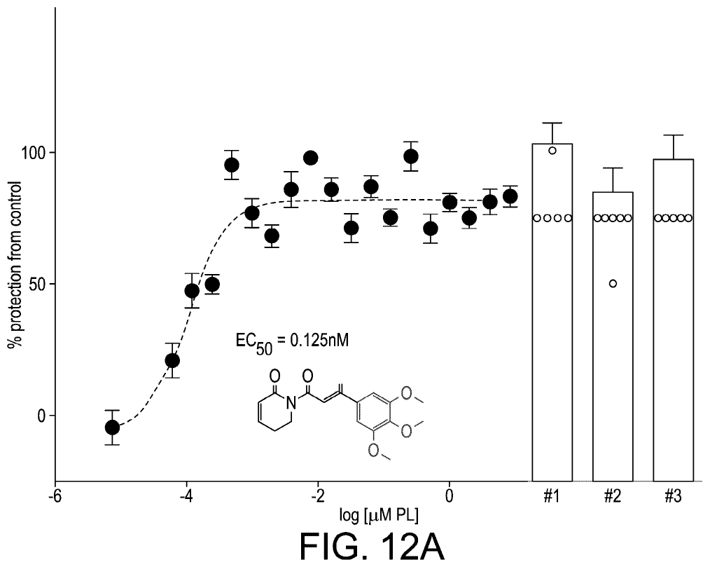

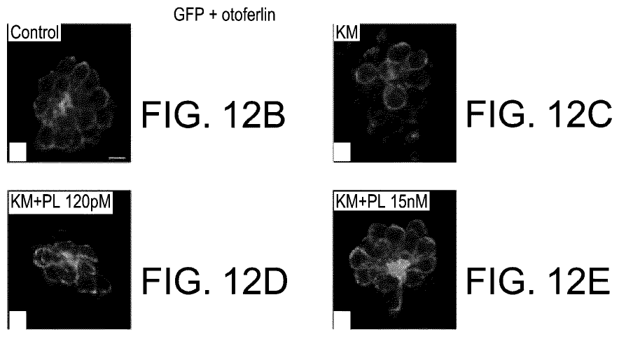

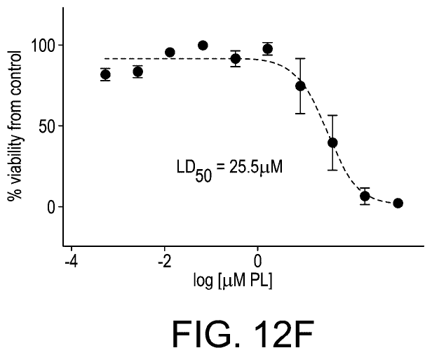

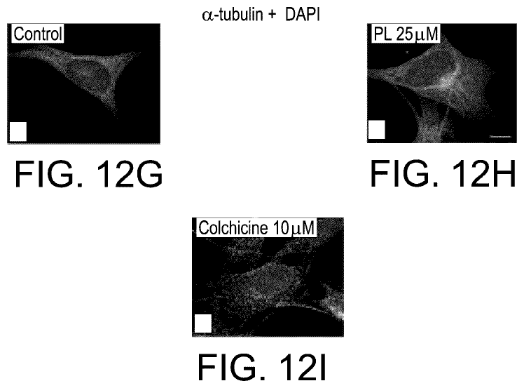

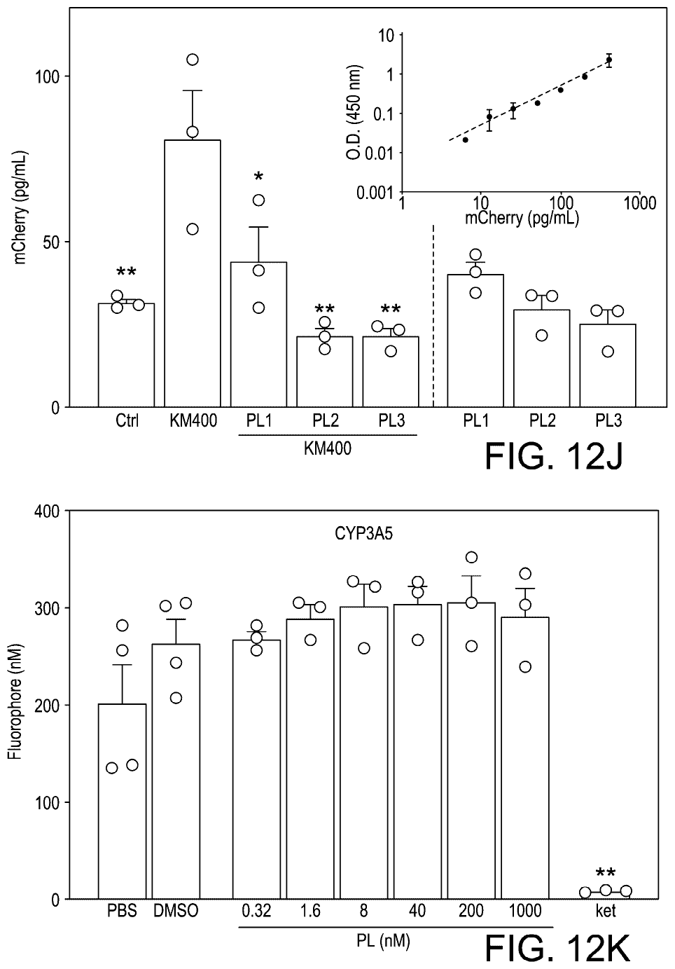

Now referring to FIGS. 12A-12K five to six days post-fertilization (dpf) zebrafish were exposed to kanamycin in the absence/presence of piperlongumine and neuromast hair cells were quantified in these animals. Zebrafish co-treated with piperlongumine (8 μM-8 pM) and kanamycin (400 μM) and immunostained for otoferlin. At least three neuromasts/fish were inspected (5-6 fish). Results are expressed as percentage of protection, with 100% representing vehicle-treated and 0% kanamycin-treated fish. piperlongumine (PL) only: #1=25 μM, #2=1 μM and #3=0.1 μM. Piperlongumine's chemical structure is shown with the colchicine motif. Piperlongumine significantly protected hair cells from kanamycin damage, with an EC₅₀ of 0.125 nM. Piperlongumine alone at high concentrations (FIG. 12A #1-#3) was not toxic to the fish or the neuromasts. The term co-treatment means that the drugs can be administered simultaneously or premixed as a formulation for administration to a subject.

FIGS. 12B-E show representative images of zebrafish neuromasts. GFP-positive hair cells immunostained for otoferlin (red).

FIG. F shows toxicity assay in HEI-OC1. PL: 1 mM-512 nM. Results are expressed as percentage of survival from control (three independent experiments). A toxicity assay performed in the mouse HC line, HEI-OC1 showed that piperlongumine can kill 50% of the cells at 25.5 μM, ˜200,000-fold higher than its effective dose. See G. Kalinec, P. Thein, C. Park, F. Kalinec, HEI-OC1 Cells as a Model for Investigating Drug-Cytotoxicity. Hear. Res.335, 105-117 (2016).

FIG.12G shows representative images from three independent experiments in HEI-OC1. Vehicle, piperlongumine 25 μM

FIG. 12H shows colchicine 10 μM

FIG. 12I shows α-tubulin immunostaining (grey).

FIG. 2J shows proximal tubule function in zebrafish (6-7 dpf) in the presence/absence of kanamycin and piperlongumine (PL1=8 μM, PL2=150 nM and PL3=150 pM). Inset. standard curve. Circles represent independent values. Because AG therapy also results in nephrotoxicity it was evaluated whether piperlongumine could protect renal function. See M. Mingeot-Leclercq, P. Tulkens, Aminoglycosides: Nephrotoxicity. Antimicrob. Agents Chemother. 43, 1003-1012 (1999). A zebrafish line Tg(Ifabp:1/2vdbp-mCherry) that expresses ½vdbp-mCherry in the liver and releases it into the circulation was used to evaluate this effect. See Z. Chen, A. Luciani, J. Mateos, G. Barmettler, R. Giles, S. Neuhauss, O. Devuyst, Transgenic Zebrafish Modeling Low-Molecular-Weight Proteinuria and Lysosomal Storage Diseases. Kidney Int. 97, 1150-1163 (2020). The presence of ½vdbp-mCherry in the fish water can be measured by ELISA and used as a proxy for proximal tubule dysfunction, See Z. Chen, A. Luciani, J. Mateos, G. Barmettler, R. Giles, S. Neuhauss, O. Devuyst, Transgenic Zebrafish Modeling Low-Molecular-Weight Proteinuria and Lysosomal Storage Diseases. Kidney Int. 97, 1150-1163 (2020). Animals were incubated with piperlongumine (EC₅₀=PL3, ×1000EC50=PL2, and ×50,000EC50=PL1) with or without kanamycin and ½vdbp-mCherry measured in the fish water. The results in FIG.12J showed that while kanamycin exposure resulted in proximal tubule dysfunction, the co-incubation with piperlongumine significantly reduced the amount of ½vdbp-mCherry released into the water. Based on these results, we concluded that proximal tubule integrity was preserved piperlongumine alone did not have any effect on renal function.

FIG. 12K shows ketoconozale: negative control. Results are presented as the average fluorophore concentration. Scale bar: B-E=5 μm, G-I=10 μm.Mean+/−SEM. Brown-Forsythe and Welch ANOVA followed by Dunnett T3 post-test for multiple comparisons. *P<0.05, **P<0.01 versus KM400 (J) or DMSO Drug-drug interactions (DDI) experiments showed that PL (1 mM-0.32 mM) did not have any effect on CYP450′s activity in vitro.

Because piperlongumine's protection of the hair cells may be the result of mechanotransduction channel blockage we performed FM1-43 uptake experiments. O. Ogun, M. Zallocchi, Clarin-1 Acts as a Modulator of Mechanotransduction Activity and Presynaptic Ribbon Assembly. J. Cell. Biol. 207, 375-391 (2014). Zebrafish were exposed to the dye with or without piperlongumine at the ˜EC₅₀ or a 100× dose. The results showed that FM1-43 was incorporated into the hair cells even at higher piperlongumine doses, confirming that piperlongumine's beneficial effect is not due to mechanotransduction blockage.

A disc diffusion assay was performed to assess the lack of piperlongumine's kanamycin interaction and thus, no interference with kanamycin's bacterial killing activity confirmed that kanamycin was able to inhibit bacterial growth even in the presence of piperlongumine. No differences were observed in the mean inhibitory growth area even at higher doses of piperlongumine (higher than the EC₅₀, 25 mM, and 100 mM) compared to kanamycin alone, which demonstrates the lack of interaction between the two molecules. This data establishes that piperlongumine can protect hair cells from aminoglycoside damage within a wide range of concentrations. The range is 0.1 pM to 25 uM. Furthermore, piperlongumine protects renal function, it has a good therapeutic index (TI>200,000), no interference with AG's anti-bacterial activity, and no drug-drug interaction.

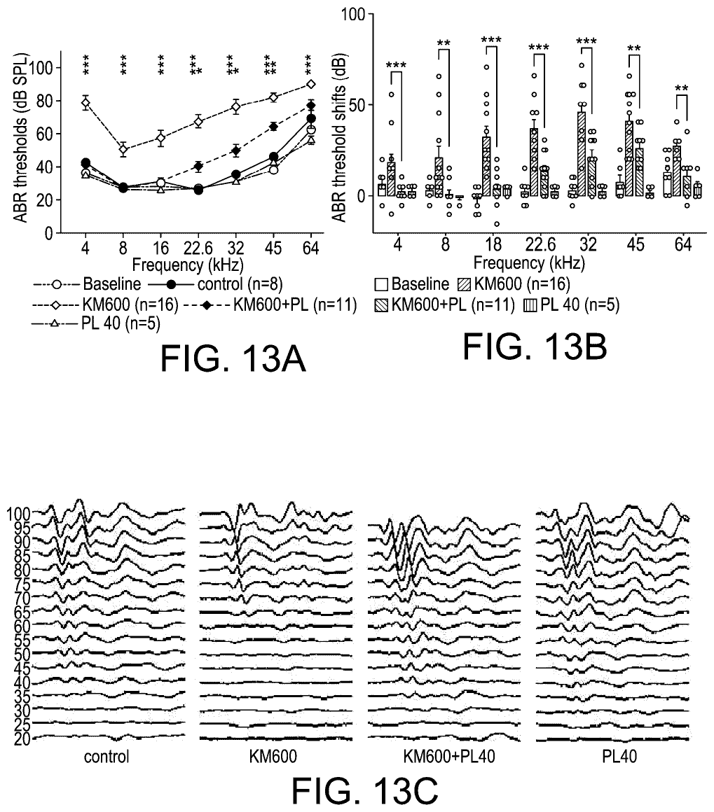

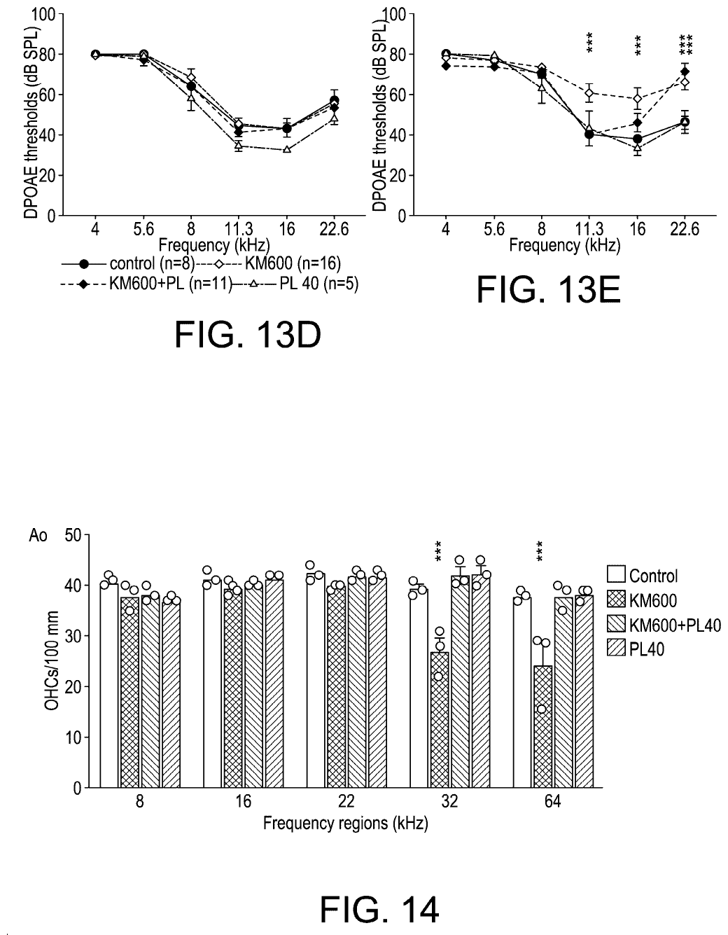

Now referring to FIGS. 13A-13E shows piperlongumine's efficacy in a mouse model for AGIHL. For this purpose, the mouse strain B6N (Cg)-Cdh23^{tm2.1/kjn} containing the corrected Cdh23 gene, preventing early onset age-related hearing loss was used for the study. Initial dosing showed that piperlongumine was well tolerated when given for 17 consecutive days at concentrations ranging from 10 mg/kg to 40 mg/kg (maximum solubility in corn oil). There were no differences in body weight between control animals receiving corn oil, and animals treated with piperlongumine.

Previous work had demonstrated that piperlongumine can cross the brain-blood barrier; mice receiving oral piperlongumine showed a peak 15-minute post-treatment with relatively steady-state levels in the brain for at least 8 hours. Thus, to test whether piperlongumine can also reach the inner ear, we injected animals intraperitoneally (IP) at 40 mg/kg and processed the inner ears for liquid chromatography with tandem mass spectrometry (LC-MS/MS) 2 hours post-treatment. A peak corresponding to piperlongumine (318.13m/z) was observed in the cochlear extracts. This same peak was observed in the reference run and in extracts from untreated animals spiked with piperlongumine, confirming the peak's identity. Cochlea extracts from animals injected with corn oil were used as the negative control. Given that piperlongumine was well tolerated and that could reach the inner ear compartment, hearing function was evaluated (auditory brainstem responses [ABRs] and distortion product otoacoustic emissions [DPOAEs]) of animals receiving kanamycin alone (600 mg/kg, twice a day, s.q for 14 days) or in combination with piperlongumine (40 mg/kg once a day, IP for 17 days starting one day before kanamycin). Body weight recorded every day during the treatment showed that animals continuously gain weight.

Now referring to FIG. 13, ABRs thresholds were elevated after kanamycin treatment at all frequencies, from 4 kHz to 64 kHz while the co-treatment with piperlongumine resulted in a significant improvement of the hearing function (reduction in threshold elevation). From 4 kHz to 16 kHz ABR thresholds were back to control values while at frequencies 22.6 kHz and higher there was a partial, although significant, recovery compared to kanamycin-only animals. The threshold shift elevations in these animals were up to 45 dB compared to vehicle controls. Conversely, threshold shift elevations for the kanamycin+PL group were significantly reduced by at least ˜15 dB when compared to the kanamycin-only group. No differences were observed between the piperlongumine-only group and the vehicle control or baseline groups black/grey), suggesting that piperlongumine, per se, does not affect the inner ear function. DPOAE thresholds, a measure of outer hair cells (OHC) function, were also significantly elevated at frequencies 11.3 kHz and higher for animals receiving kanamycin piperlongumine co-treatment significantly reduced kanamycin's effect at 11.3 kHz and16 kHz but not at 22.6 kHz, this latter result was in accordance with the ABR recordings, where there was partial protection of piperlongumine at frequencies above 22.6 kHz.

Because systemically administered aminoglycoside enters the cochlea mostly through the stria vasculari, the endocochlear potential of the endolymphatic compartment was measured to assess whether the stria vascularis was affected by the presence of aminoglycosides. No differences were observed among the treatments, suggesting that the chronic administration of AGs does not severely affect the stria vascularis function, and also reinforcing the notion that the sensory epithelia is the main target for aminoglycoside ototoxicity. These results confirm previous work from Hirose et al., in which the acute administration of kanamycin and furosemide did not produce any significant drop in the EP values. Overall, the hearing test results suggest that piperlongumine can be protective against AG ototoxicity in mammals. No sex differences were observed in terms of body weight gain, treatment and recovery, and level of piperlongumine protection.

Now referring to FIG. 14, to correlate the hearing tests with inner ear morphology, the organs of Corti from the four groups were stained for the HC marker, myosin-7A, and the presynaptic ribbon marker, Ctbp2. The OHCs were imaged and quantified at 5 frequency regions: 8 kHz-64 kHz. Kanamycin treatment resulted in a significant loss of OHCs at the 32 kHz and 64 kHz regions while piperlongumine treatment prevented the OHCs from dying. At lower frequencies and despite the significant increase in ABR thresholds due to kanamycin treatment, OHCs were still present and functional which suggests that there is additional damage to the sensory epithelia that renders a decrease in auditory function. No deleterious effect was observed in the organ of Corti from animals treated with piperlongumine alone.

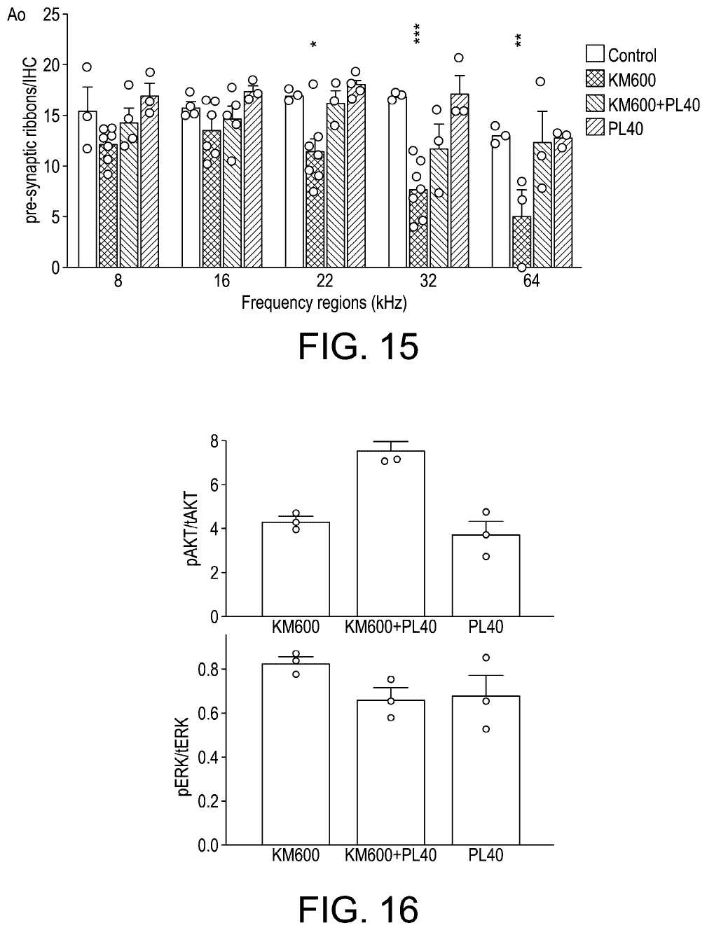

Now referring to FIG. 15, the quantification of the pre-synaptic ribbons in the kanamycin-only treated group showed a reduction in their number for the 22 kHz through 64 kHz frequency regions. Conversely, piperlongumine co-treatment resulted in full recovery in their numbers. Similarly to the OHC results, piperlongumine alone did not have any toxic effect on the Ctbp2-positive puncta,

An increase in ABR thresholds can be the result of a decrease in the number of synaptic contacts between the inner HCs (IHCs) and the afferent fibers, and this can be reflected in the amplitudes and latencies of the ABR peaks. Thus, as part of our hearing assessment and because PL treatment did not fully restore inner ear function although OHCs and pre-synaptic structures were protected, we analyzed wave I amplitudes and latencies. When analyzing the ABR wave-I suprathreshold in the kanamycin-only group we observed a decrease in wave-I amplitudes and an increase in latencies at all the tested frequencies (16 kHz-64 kHz). For the wave I amplitudes at 64 kHz, although the decrease was not significant. A trend was observed in the kanamycin-treated animals. The administration of piperlongumine did not fully recover kanamycin's deleterious effect at frequencies between 22.6 kHz to 64 kHz (Compared to vehicle or-piperlongumine alone wave I amplitudes were still significantly low, and latencies were significantly delayed in the kanamycin+piperlongumine group. Only at 22.6 kHz, we observe a tendency to counteract kanamycin toxicity, but for frequencies above 22.6 kHz, piperlongumine was unable to fully restore inner ear function. These observations correlate with the modest (but significant) threshold elevation in the ABR recordings after piperlongumine co-treatment. The 16 kHz region (was used as a positive control since it shows full protection (ABR thresholds similar to controls) after piperlongumine co-treatment. Given the lack of recovery of wave I amplitudes and latencies after piperlongumine co-treatment despite the full recovery of the Ctbp2-positive puncta (we decided to look at post-synaptic structures. For this purpose, organs of Corti were immunostained for myosin7A and the neuronal fiber marker, neurofilament H (The intricate network of neuronal fibers innervating the base of the IHCs was analyzed with the Imaris filament tracer tool. No differences were observed among the treatments. Kanamycin alone did not affect the neuronal array: covered area, volume of the fiber, length, and density were not significantly different among the four groups in summary, although piperlongumine protected outer hair cells and pre-synaptic ribbons from kanamycin ototoxicity, wave I amplitudes, and latencies were not fully reversed.

Piperlongumine's role in TRPV1/AKT1 pathway

To characterize the mechanism(s) through which piperlongumine can exert its beneficial effect in the inner ear, a kinome profile analysis of serine/threonine kinases (STKs) was performed. The inner ears were microdissected ten days after receiving the first kanamycin dose, which allowed the emergence of hearing loss (significant elevation of ABR thresholds, without hair cell death. The identification of the potential STKs was performed with the PamGene (34) kinome platform that contains.

Now referring to FIG. 16 samples were run in technical triplicates and the differentially phosphorylated peptides were processed using three different deconvolution strategies by using this approach, the AKT family members as candidate “hits” contributing to the phosphorylation signal seen across the reporter peptides on the STK chip Immunoblot experiments confirmed the results obtained with the kinome. AKT1 phosphorylation was significantly increased in the organ of Corti lysates from animals co-treated with kanamycin and piperlongumine compared to the rest of the groups. ERK activation was used as a control to reinforce the positive correlation between the kinome results and the immunoblots. There was no differential activation of the ERK family members assessed by both approaches which demonstrates the reliability of the kinome profile.

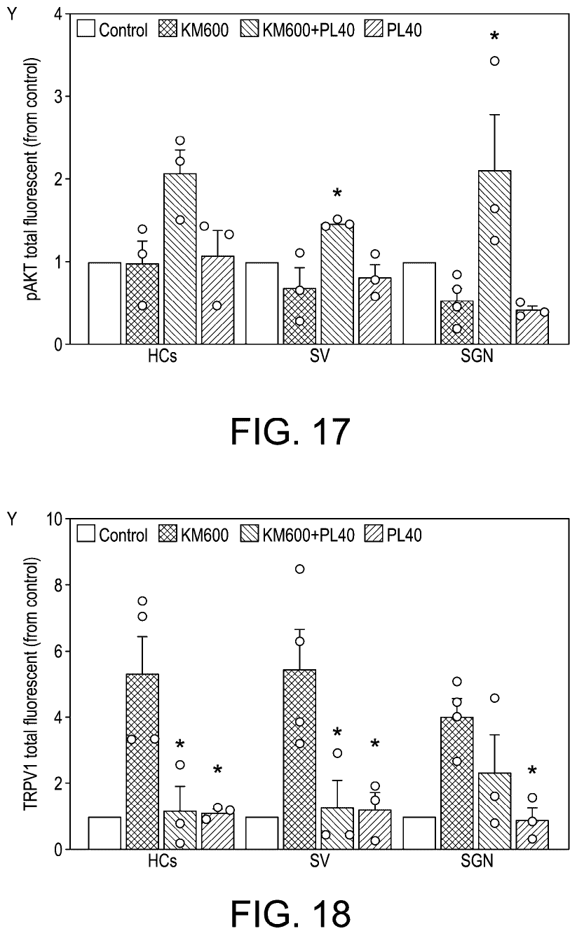

Now referring to FIG. 17, the pAKT1 immunohistochemistry results confirmed the kinome and the immunoblot analysis and suggest that the regulation of AKT1 may be important for inner ear protection. During inflammation and aminoglycoside insult, cochlear expression of the transient receptor vanilloid 1 (TRPV1) channel is upregulated in the inner ear, including the stria vascularis, spiral ganglion neurons (SGNs), and the hair cells. This increment in TRPV1 results in an exacerbation of AG ototoxicity due to an increase in aminoglycoside uptake, with TRPV1 playing a key role during this process. In addition, TRPV1 activation can impact mitochondrial homeostasis and ROS production by preventing AKT activation. More importantly piperlongumine can allosterically antagonize TRPV channels' activity. Conde, R. Pumroy, C. Baker, T. Rodrigues, A. Guerreiro, B. Sousa, M.C. Marques, B.P. de Almeida, S. Lee, E. Leites, D. Picard, A. Samanta, S. Vaz, F. Sieglitz, M. Langini, M. Remke, R. Roque, T. Weiss, M. Weller, Y. Liu, S. Han, F. Corzana, V. Morais, C. Faria, T. Carvalho, P. Filippakopoulos, B. Snijder, N. Barbosa-Morais, V. Moiseenkova-Bell, G. Bernardes, Allosteric Antagonist Modulation of TRPV2 by Piperlongumine Impairs Glioblastoma Progression. ACS Cent. Sci. 7, 868-881 (2021).

Now referring to FIG. 18, is can be seen that piperlongumine treatment results in TRPV1 downregulation Chronic administration of kanamycin (or other toxins) increased TRPV1 expression in the three compartments (stria vascularis “SV”, hair cells “HCs” and spiral ganglion neuron “SGN) while piperlongumine co-treatment resulted in a reduction of TRPV1 expression along the cochlea piperlongumine (-only treatment resulted in an increase in TRPV1 expression in supporting cells (i.e., inner phalangeal or inner border cells). The quantification of the total fluorescence intensity of the regions of interest from at least three different animals demonstrated the increase in TRPV1 staining due to aminoglycoside treatment and the reduction during PL therapy. Altogether these results suggest that piperlongumine's beneficial effect may be mediated, at least in part, by down-regulation and/or desensitization of TRPV1 and thus, a reduction in aminoglycoside uptake. Quantification is presented as the ratio of fluorescence for each region relative to the total fluorescence. Mean+/−SEM. Circles represent values from different animals. Three independent samples were analyzed. One-way ANOVA followed by Dunnett post-test for multiple comparisons. *P<0.05 versus kanamycin.

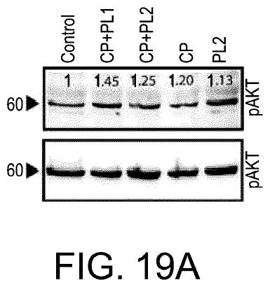

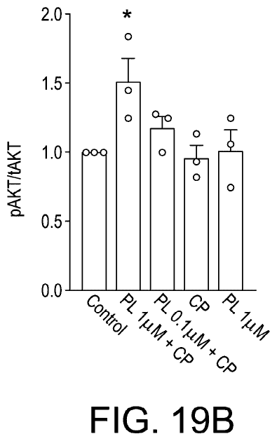

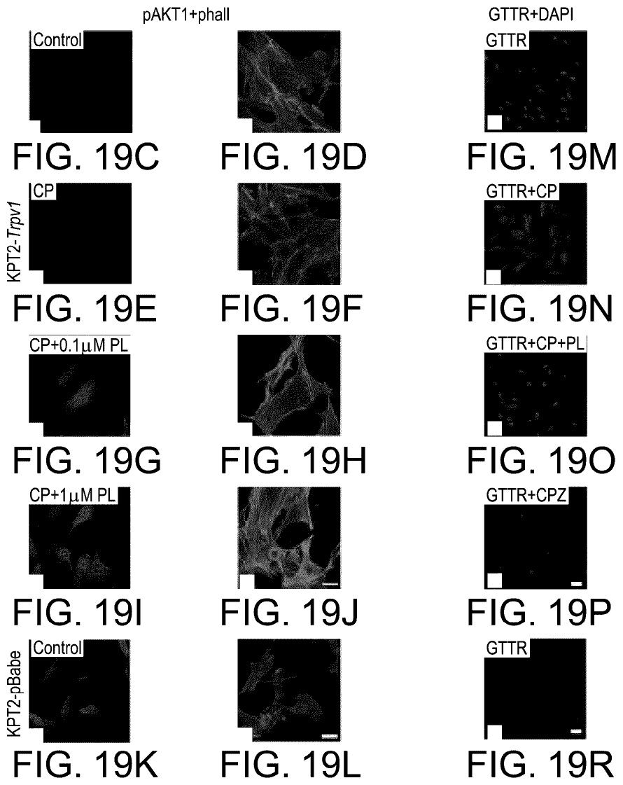

Now referring to FIGS. 19A-19R, piperlongumine regulates aminoglycoside uptake via TRPV1 interaction. AKT1 phosphorylation was measured in KPT2-Trpv1 cells incubated with media alone, CP 3 μM, PL 1 μM, or PL+CP (PL1=1 μM or PL2=100 nM). Only when the cells were co-incubated with PL1+CP, AKT1 was activated. There was a modest albeit significant increase in AKT1phosphorylation. CP, PL, and PL2 (100 nM) +CP did not increase pAKT1 levels. Finally, to assess the relationship between PL, TRPV1, AKT1, and aminoglycoside uptake, we used a proximal tubular epithelial cell line stably expressing TRPV1 (KPT2-Trpv1) or a retroviral control vector (KPT2-pBabe) (37). These lines have already been characterized and used to test the role of TRPV1 during aminoglycoside uptake (37). AKT1 phosphorylation was measured in KPT2-Trpv1 cells incubated with media alone, CP 3 μM, PL 1μM, or PL+CP (PL1=1 μM or PL2=100 nM). Only when the cells were co-incubated with PL1+CP, AKT1 was activated. There was a modest albeit significant increase in AKT1 phosphorylation. CP, PL, and PL2 (100 nM) +CP did not increase pAKT1 levels. AKT1 phosphorylation was also assessed in the two cell lines by immunofluorescence Similarly to the immunoblot results, we observed a positive pAKT1 signal when cells were co-incubated with PL+CP or PL-only No immunofluorescence was detected in non-stimulated cells (Control), or cells incubated with CP-only. Moreover, in the KPT2-pBabe cells (transfected with the empty vector) we observed an increase of pAKT1, suggesting that indeed, TRPV1 inhibits AKT1 activity. Lastly, we performed gentamicin Texas-Red (GTTR) uptake experiments. Under basal conditions there was minimal incorporation of GTTR by the KPT2-Trpv1. This changed (increased) when the cells were incubated with GTTR in the presence of CP, confirming the involvement of TRPV1 during aminoglycoside s incorporation. Conversely, when the cells were co-incubated with CP+PL, GTTR uptake was impaired and similar to the results obtained with the TRPV1 antagonist (capsazepine, CPZ. The KTP2-pBabe cells did not show any detectable GTTR incorporation. In conclusion, this data provides compelling evidence of a regulatory axis involving TRPV1 activation, AKT1 dephosphorylation, and aminoglycoside uptake. In summary, piperlongumine's beneficial effect is via TRPV1 modulation (downregulation and/or desensitization). This reduction in TRPV1′s overall activity results in AKT1 activation which in turn, allows the preservation of the redox state in the inner ear and the protection of hearing function.

All publications, patents and patent applications mentioned in this specification are herein incorporated by reference to the same extent as if each individual publication, patent, or patent application was specifically and individually indicated to be incorporated by reference.

While the invention has been described with reference to details of the illustrated embodiments, these details are not intended to limit the scope of the invention as defined in the appended claims. The embodiment of the invention in which exclusive property or privilege is claimed is defined as follows:

What is claimed:

1. A method to prevent aminoglycoside toxicity comprising systemically co-administering to an animal or human, an aminoglycoside and an effective amount of piperlongumine to prevent aminoglycoside toxicity.

2. The method of claim 1, wherein the aminoglycoside toxicity affects ear hair cells.

3. The method of claim 1, wherein the aminoglycoside toxicity affects renal function.

4. The method of claim 2, wherein the concentration of the piperlongumine ranges from 0.1 pM to 25 uM.

5. A method of preventing aminoglycoside toxicity, the method comprising administering a therapeutically effective amount of an active agent to downregulate TRPV1, or a pharmaceutically acceptable salt thereof and an aminoglycoside, wherein the active agent is selected from the group consisting of: piperlongumine; a derivative of piperlongumine or a pharmaceutically acceptable salt thereof.

6. The method of claim 5, wherein the aminoglycoside toxicity affects ear hair cells.

7. The method of claim 5, wherein the aminoglycoside toxicity affects renal function.

8. The method of claim 5, wherein the concentration of the active agent ranges from 0.1 pM to 25 uM.

9. A formulation for systemic administration to a subject comprising: an effective amount a derivative of piperlongumine to prevent aminoglycoside toxicity in a pharmaceutically acceptable carrier.

10. The formulation of claim 9, wherein the derivative of piperlongumine is comprised of the formula:

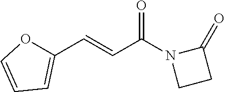 [SMILES: O=C(/C=C/c1ccco1)N1CCC1=O]

11. The formulation of claim 9, wherein the derivative of piperlongumine is comprised of the formula:

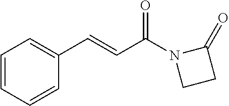 [SMILES: O=C(/C=C/c1ccccc1)N1CCC1=O]

12. The formulation of claim 9, wherein the derivative of piperlongumine is comprised of the formula (C16H15F2NO4):

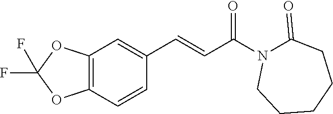 [SMILES: O=C(/C=C/c1ccc2c(c1)OC(F)(F)O2)N1CCCCCC1=O]

13. The formulation of claim 9, wherein the derivative of piperlongumine (PG 54 (SHJ-54)) is comprised of the formula (C15H13F2NO4):

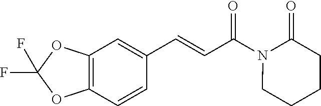 [SMILES: O=C(/C=C/c1ccc2c(c1)OC(F)(F)O2)N1CCCCC1=O]

14. A kit comprising: an active agent, wherein the active agent is piperlongumine, a derivative of piperlongumine, or a pharmaceutically acceptable salt thereof; and one or more of:
at least one antibiotic; or
at least one cancer drug; and
instructions for preventing a hearing impairment.

15. The kit of claim 14, wherein the at least one antibiotic is an aminoglycoside.

16. The kit of claim 15, wherein the at least one antibiotic is neomycin.

17. The kit of claim 15, wherein the at least one antibiotic is kanamycin.

18. The kit of claim 14, wherein the at least one cancer drug is cisplatin.

19. A kit comprising: an active agent, wherein the active agent is piperlongumine, a derivative of piperlongumine, or a pharmaceutically acceptable salt thereof; and one or more of: at least one aminoglycoside antibiotic; and, instructions for preventing aminoglycoside toxicity.

20. The kit of claim 19, wherein the at least one aminoglycoside antibiotic is selected from the group consisting of: gentamicin, tobramycin, amikacin, neomycin, netilmicin, plazomicin, paromomycin, streptomycin and kanamycin.
